(19) United States
(12) Patent Application Publication
(10) Pub. No.: US 2022/0002808 A1
(43) Pub. Date: Jan. 6, 2022

(54) ARTIFICIAL INTELLIGENCE AND BLOOD EPIGENOMIC ANALYSIS FOR ALZHEIMERS DISEASE

(71) Applicant: William Beaumont Hospital, Royal Oak, MI (US)

(72) Inventors: Ray BAHADO-SINGH, Grosse Pointe Shores, MI (US); Sangeetha VISHWESWARAIAH, Clawson, MI (US); Uppala RADHAKRISHNA, Troy, MI (US); Stewart Francis GRAHAM, Berkley, MI (US)

(21) Appl. No.: 17/366,959

(22) Filed: Jul. 2, 2021

Related U.S. Application Data
(60) Provisional application No. 63/047,427, filed on Jul. 2, 2020.

(51) Int. Cl. C12Q 1/6883; G16B 40/00; G16H 50/20; G06N 3/08; G06N 20/10; G06N 20/20

(52) U.S. Cl. CPC C12Q 1/6883; G16B 40/00; G16H 50/20; C12Q 2600/154; G06N 20/10; G06N 20/20; C12Q 2600/118; G06N 3/08

(57) ABSTRACT

An Artificial intelligence-based method for diagnosing Alzheimer's Disease or determining susceptibility to Alzheimer's Disease includes a step of obtaining a blood sample from a target subject (e.g., a human). The degree of methylation in one or a plurality of Alzheimer indicators genes is identified for leukocytes in the blood sample. Each Alzheimer indicator gene is identified as being an indicator of the presence of or risk of developing Alzheimer's Disease. Characteristically, the at least one or the plurality of Alzheimer indicators genes have been identified by a machine learning technique or by logistic regression. Finally, the target subject is identified as being at risk for Alzheimer's Disease if the amount of methylation of one or more Alzheimer indicators genes differs from the amount of methylation established in control subjects (for the same genes) not having Alzheimer's Disease by a predetermined amount.

### CROSS-REFERENCE TO RELATED APPLICATIONS

This application claims the benefit of U.S. provisional application Ser. No. 63/047,427 filed Jul. 2, 2020, the disclosure of which is hereby incorporated in its entirety by reference herein.

### TECHNICAL FIELD

In at least one aspect, the present invention is related to methods for diagnosing Alzheimer's Disease in a subject.

### BACKGROUND

Alzheimer's Disease (AD) is the most common form of dementia accounting for 50-70% of such cases. The disorder causes significant mental disabilities, and manifest profound behavioral changes, physical disabilities and progressive impairment of social skills. Globally, in 2015 close to 47 million individuals had the disorder. As the population ages, estimates are that 75 million will be affected world-wide in 2030 with a projected rise to 131 million in 2050 (Prince et al. 2015b). The World Health Organization in a report released in April 2012 entitled ‘Dementia: A Public Health Priority’ has declared AD a global health priority (Wortmann 2012).

Precision Medicine in AD will be driven by the integration of breakthrough technologies such as systems biology, genomics, big data science and blood-based biomarkers (Hampel et al. 2017). Genome-wide DNA methylation analysis of blood leukocytes is a new and developing discipline devoted to the identification of significant genes and putative biomarkers in brain disorders (Berkel & Pandey 2017). Brain disorders studied include schizophrenia (Song et al. 2014), major depression (Liu et al. 2014) and drug addiction (Berkel & Pandey 2017). (Bahado-Singh et al. 2019a). Currently there is almost no information on AD.

The mechanisms leading to AD development largely remain a mystery. While several different single-gene mutations on chromosomes 21, 14, and 1 (OMIM: #104300) including mutations in the apolipoprotein E (APOE) gene confers some risk (Liu et al. 2013a) so far the cumulative contribution of genetic factors in sporadic AD appears to be small. The pathogenesis of late-onset Alzheimer disease likely includes a major contribution from environmental, and lifestyle factors (Daviglus et al. 2010). Epigenetics is the mechanism by which such environmental and life-style factors modulate gene expression and thus epigenetics appears likely to be important in AD development (Grinan-Ferre et al. 2018). DNA methylation changes in the brain have been found to be an early feature of AD (De Jager et al. 2014). The epigenetic mechanisms of AD is currently poorly understood however.

While not definitive, existing evidence suggest that there is significant alterations of peripheral leucocytes in AD. These include T-lymphocytes (Town et al. 2005), B-lymphocytes (Richartz-Salzburger et al. 2007), polymorphonuclear leucocytes (Rezai-Zadeh et al. 2009) and monocytes and macrophages (Kusdra et al. 2000) changes. Alterations in DNA methylation have also been reported in peripheral whole blood of AD subjects (Li et al. 2016). Identifying specific genes that are epigenetically dysregulated could provide insight into the mechanism of AD development.

Artificial Intelligence (AI) is rapidly transforming modern life in areas as diverse as face recognition and robotics. Machine Learning (ML) is a branch of AI and Deep Learning (DL) is the latest developing branch of ML. ML involves learning by computers that do not require any or only minimal explicit programing by humans. An area of current interest is the use of AI for patient categorization and diagnosis based on review of electronic health records. There is early interest in the use of DL for analysis and prediction using biologic big-data such as genomics (Mamoshina et al. 2016; Ching et al. 2018), epigenomics (Bahado-Singh et al. 2019a) and metabolomics (Bahado-Singh et al. 2018; Alpay Savasan et al. 2019; Bahado-Singh et al. 2019b) to understand and accurately predict human disorders.

DNA methylation analysis of peripheral leucocytes to generate potential biomarkers for brain disorders is a newly developing field. It opens the possibility of non-invasive (blood test) evaluation of the brain. The applicability of this approach to Alzheimer's disease (AD), the most common form of dementia, is currently unknown.

Accordingly, there is a need to develop new and more accurate methods for diagnosing Alzheimer's Disease.

### SUMMARY

The present invention solves one or more problems of the prior art by providing in at least one aspect, a method in which DL and several other ML techniques are combined with DNA methylation analysis of blood leucocytes for AD detection.

In another aspect, molecular pathway analysis is applied to investigate the potential epigenetic mechanisms in late-onset AD.

In another aspect, a method for diagnosing Alzheimer's Disease or determining susceptibility to Alzheimer's Disease is provided. The method includes a step of obtaining a blood sample from a target subject (e.g., a human). The degree of methylation of cytosine (CpG) loci in one or a plurality of Alzheimer indicators genes is identified in leukocytes in the blood sample. Each Alzheimer indicator gene or more precisely CpG locus in a given gene is identified as being an indicator of the presence of or risk of developing Alzheimer's Disease. Characteristically, at least one or the plurality of Alzheimer indicators genes have been identified by a machine learning technique or by logistic regression. Finally, the target subject is identified as being at risk for Alzheimer's Disease if the amount of methylation of one or more Alzheimer indicators genes differs from the amount of methylation established in control subjects (for the same CpG loci in the same genes) not having Alzheimer's Disease by a predetermined amount.

In another aspect, a method for diagnosing Alzheimer's Disease or determining susceptibility to Alzheimer's Disease is provided. The method includes a step of obtaining a blood sample from a target subject which can include blood spot on a filter paper obtained from a finger stick or blood drops from finger stick placed directly into a receptacle for subsequent DNA extraction. Gene methylation analysis of leucocytes in the blood sample is performed. A trained neural network is applied to determine if the target subject is at risk for or has Alzheimer's disease, the trained neural network having been trained from genome-wide methylation test sets that include a first group of testing subjects having Alzheimer's disease and a second group of test subjects not having Alzheimer's disease.

In another aspect, an AI system for calculating risk of AD based on leucocyte DNA methylation analysis is provided. The AI system includes a computer processor executing the steps of the method comprising:

obtaining a blood sample from a target subject;

identifying the degree of methylation in one or a plurality of Alzheimer indicators genes in the blood sample, each Alzheimer indicators gene identified as being an indicator of the presence of or risk of developing Alzheimer's Disease, the plurality of Alzheimer indicators genes having been identified by a machine learning technique or by logistic regression; and

identifying the target subject as being at risk for Alzheimer's Disease if the amount of methylation of one or more Alzheimer indicator genes differs from the amount of methylation established in control subjects not having Alzheimer's Disease by a predetermined amount.

In another aspect, the methods and AI systems are applied to calculate a risk of Alzheimer's Disease among patients having mild cognitive impairment.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements and wherein:

FIG. 1: Epigenetically dysregulated molecular pathways in AD.

FIG. 2: Epigenetically dysregulated disease pathways in AD.

FIG. 3. Partial Least Square Discriminant Analyses with intragenic markers.

FIG. 4. Table 1: Top 25 differentially methylated CpG markers based on FDR-p values with gene IDs, chromosome location, fold change, AUC and percentage of methylation difference for each CpGs identified in the study.

FIG. 5. Table 2: Alzheimer's disease prediction based on Intragenic CpG markers only (threshold: Benjamini-Hochberg FDR p-value<0.05)

FIG. 6. Table 3: Alzheimer's disease prediction based on Intragenic CpG markers only (stringent threshold: p-value<5×10⁻⁸* used for Genome-wide association studies)

FIG. 7. Table 4: Clinical and demographic characteristics compared between AD cases and normal control subjects

FIGS. 8A, 8B, 8C, and 8D. Table 5: Additional significant differentially methylated CpG markers based on FDR-p values<0.05 with gene IDs, chromosome location, fold change, AUC and percentage of methylation difference for each CpGs identified in the study.

FIG. 9. Tables 6: Top 25 extragenic markers based on FDR p-value<0.05

FIG. 10. Tables 7: Top 25 extragenic markers based on stringent p-5×10⁻⁸.

FIG. 11. Tables 8: Alzheimer's disease prediction based on Intragenic CpG markers (threshold: FDR p-value<0.05) combined with Clinical and Demographic markers.

FIG. 12. Table 9: Alzheimer's disease prediction based on Intragenic CpG markers (threshold: p-value<5×10^{-8*}) combined with Clinical and Demographic Markers.

FIG. 13. Table 10: Alzheimer's disease prediction based on Extragenic CpG markers (threshold: FDR p-value<0.05).

FIG. 14. Table 11: Alzheimer's disease prediction based on Extragenic CpG markers (threshold: p-value<5×10^{-8*}).

FIG. 15. Table 12: Differentially methylated genes enriched molecular pathways in Alzheimer's disease.

FIG. 16. Table 13: Differentially methylated genes enriched in Disease pathways of Alzheimer's disease

FIGS. 17A and 17B. Table 14: List of genes that were found to be significantly differentially methylated and which references indicate are currently known or plausible linked to AD.

FIGS. 18A, 18B, and 18C. Table 15: AD blood-Group-152 markers-FDR p<0.05.

FIG. 19. Table 16: AD blood-Group-33 markers-p-value<5×10⁻⁸.

FIG. 20. Table 17: AD blood-Group-10 CpG leucocyte DNA markers—in genes previously reported to differentially expressed in the brain of AD patients.

FIG. 21. A block diagram of a computing system that can be used to implement the method for diagnosing Alzheimer's Disease or determining susceptibility to Alzheimer's Disease and related training.

### DETAILED DESCRIPTION

Reference will now be made in detail to presently preferred compositions, embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

The processes, methods, or algorithms disclosed herein can be deliverable to/implemented by a processing device, controller, or computer, which can include any existing programmable electronic control unit or dedicated electronic control unit. Similarly, the processes, methods, or algorithms can be stored as data and instructions executable by a controller or computer in many forms including, but not limited to, information permanently stored on non-writable storage media such as ROM devices and information alterably stored on writeable storage media such as floppy disks, magnetic tapes, CDs, RAM devices, and other magnetic and optical media. The processes, methods, or algorithms can also be implemented in a software executable object. Alternatively, the processes, methods, or algorithms can be embodied in whole or in part using suitable hardware components, such as Application Specific Integrated Circuits (ASICs), Field-Programmable Gate Arrays (FPGAs), state machines, controllers or other hardware components or devices, or a combination of hardware, software and firmware components.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1 to 2.1 the following numbers 1.2, 1.3, 1.4, 1.5, 1.6, 1.7, 1.8, 1.9, and 2.0 can be selected as lower or upper limits.

The dosage of any therapeutic agent administered to treat Alzheimer's Disease is dependent on numerous factors such as the particular species, its weight, the level of impairment, the desired degree of treatment, and the individual itself. Dosages can be readily determined by one skilled in the art by routine tests, for example time/serum level measurements, dose/response curves, etc. The dosages are in particular easy to range, as numerous monoamine transport-affecting drugs are commercially available, have extensive in vitro and in vivo results presented in the literature, or are in clinical trials.

Throughout this application, where publications are referenced, the disclosures of these publications in their entireties are hereby incorporated by reference into this application to more fully describe the state of the art to which this invention pertains.

### Abbreviation

“AD” means Alzheimer's Disease.

“AI” means artificial intelligence.

“DL” means Deep Learning.

“ML” means machine learning.

In an embodiment, a method for diagnosing Alzheimer's Disease or determining susceptibility to Alzheimer's Disease is provided. The method includes a step of obtaining a blood sample from a target subject (e.g., a human). The degree of methylation in one or a plurality of Alzheimer indicators genes is identified in the DNA of leukocytes in the blood sample. Each Alzheimer indicator gene is identified as being an indicator of the presence of or risk of developing Alzheimer's Disease. Characteristically, the at least one or the plurality of Alzheimer indicators genes have been identified by a machine learning technique or by logistic regression. Finally, the target subject is identified as being at risk for Alzheimer's Disease if the amount of methylation of one or more Alzheimer indicators genes differs from the amount of methylation established in control subjects (for the same genes) not having Alzheimer's Disease by a predetermined amount. In a refinement, the predetermined amount is at least a 30 percent difference in the amount of methylation as compared to control subjects (for corresponding genes between target subject and controls). The percent different is (|control−target subject|/control)*100%). In other refinements, the predetermined amount is at least, in increasing order of preference, 30 percent, 50 percent, 100 percent or 200 percent difference in the amount of methylation as compared to control subjects (for corresponding genes between target subject and controls).

In a variation, the method further includes a step of treating the target subject for Alzheimer's Disease is the target subject is identified as being at risk. Examples of agents that can be used to treat Alzheimer's Disease include, but are not limited to, cholinesterase inhibitors (e.g., donepezil, rivastigmine, and galantamine), mematine (e.g., 4 mg to 25 mg/day), Vitamin E at a dose of about 1000 IU to 3000 IU per day (e.g., 2000 IU per day), and aducanumab. Aducanumab can be administered by intravenous (IV) infusion every four weeks (e.g., 1 mg/kg to 15 IV over one hour spread over 36 weeks or more). In a refinement, the target subject is treated in clinical trial for Alzheimer's Disease if the target subject is identified as being at risk in a clinical trial.

As set forth above, at least one or the plurality of Alzheimer indicators genes have been identified by a machine learning technique or by logistic regression is identified by a machine learning technique. A particularly useful type of machine learning technique is a neural network method. Additional examples of machine learning techniques that can be applied include, but are not limited to support vector machine (SVM), a Generalized linear Model (GLM), Prediction Analysis for Microarrays (PAM), Random Forest (RF) and Linear Discriminant Analysis (LDA). Each of these approaches can be used to estimate AD risk.

In another variation, at least one gene in the plurality of Alzheimer indicators genes are hypomethylated in target subjects having or at risk for Alzheimer's Disease as compared to control subjects. Examples of such hypomethylated genes or more precisely reduced methylation of the predictive cytosine locus in that gene include, but are not limited to PLVAP, KCNH2, TSTD3, SARM1, CTHRC1, TRAM1L1, GUSBL2, LOC731275, ZNF254 and TRIM6. In this regard, it should be appreciated that a target subject can be evaluated for one or any combination of these genes.

In another variation, at least one gene or more precisely the predictive cytosine locus in that gene in the plurality of Alzheimer indicators genes are hypermethylated in target subjects having or at risk for Alzheimer's Disease as compared to control subjects. Examples of such hypermethylated genes or more precisely the predictive cytosine locus in that gene include, but are not limited to RNF5P1, RNF5, AGPAT1, GRB10, MIB2, WNT9B, MAF, THAP4, KCNK5 and KIF26A. In this regard, it should be appreciated that a target subject can be evaluated for one or any combination of these genes.

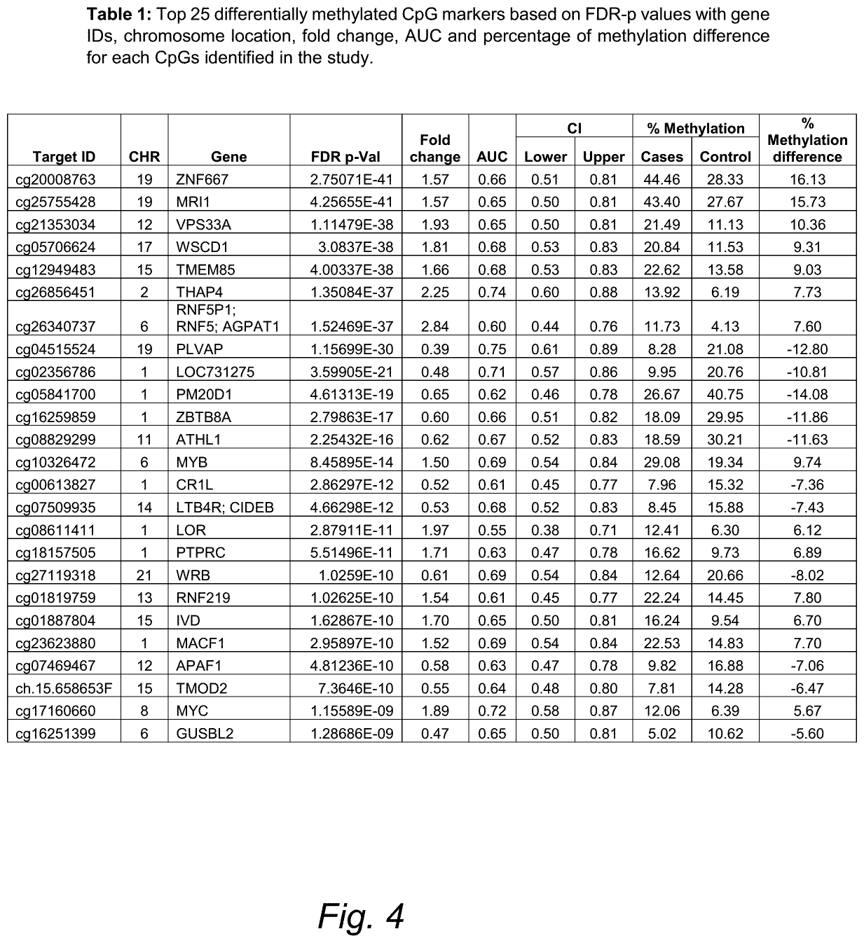

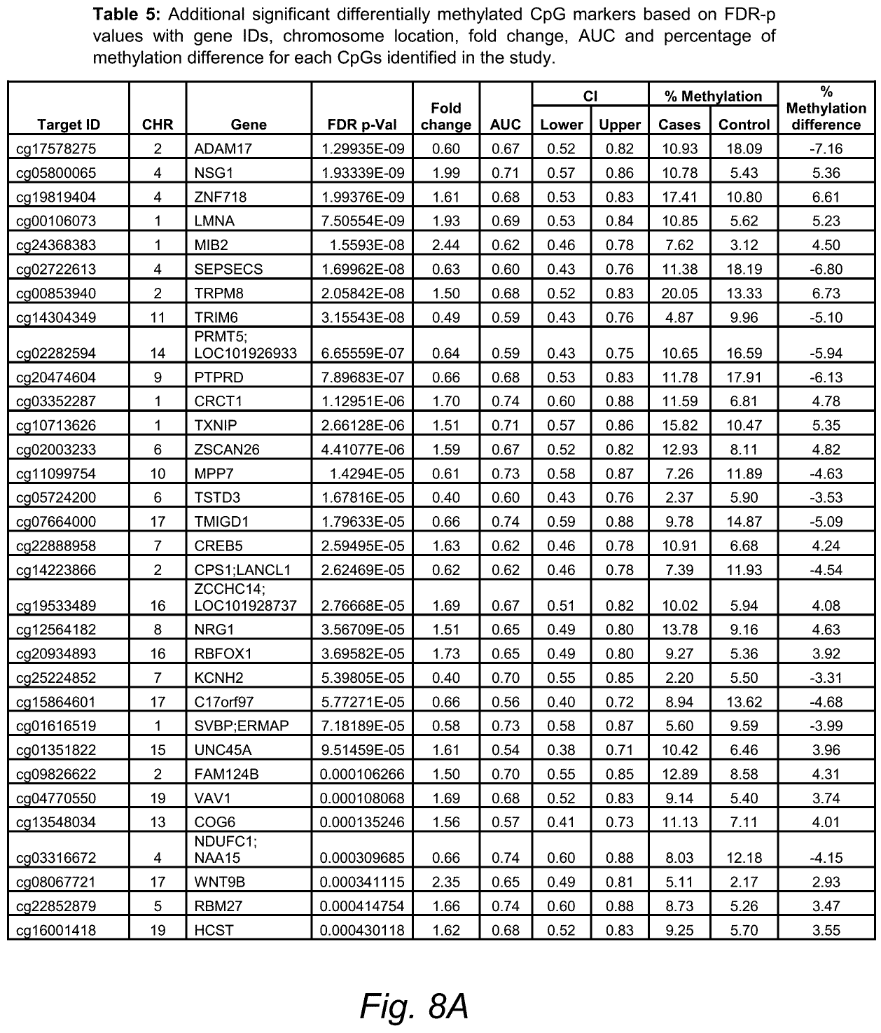

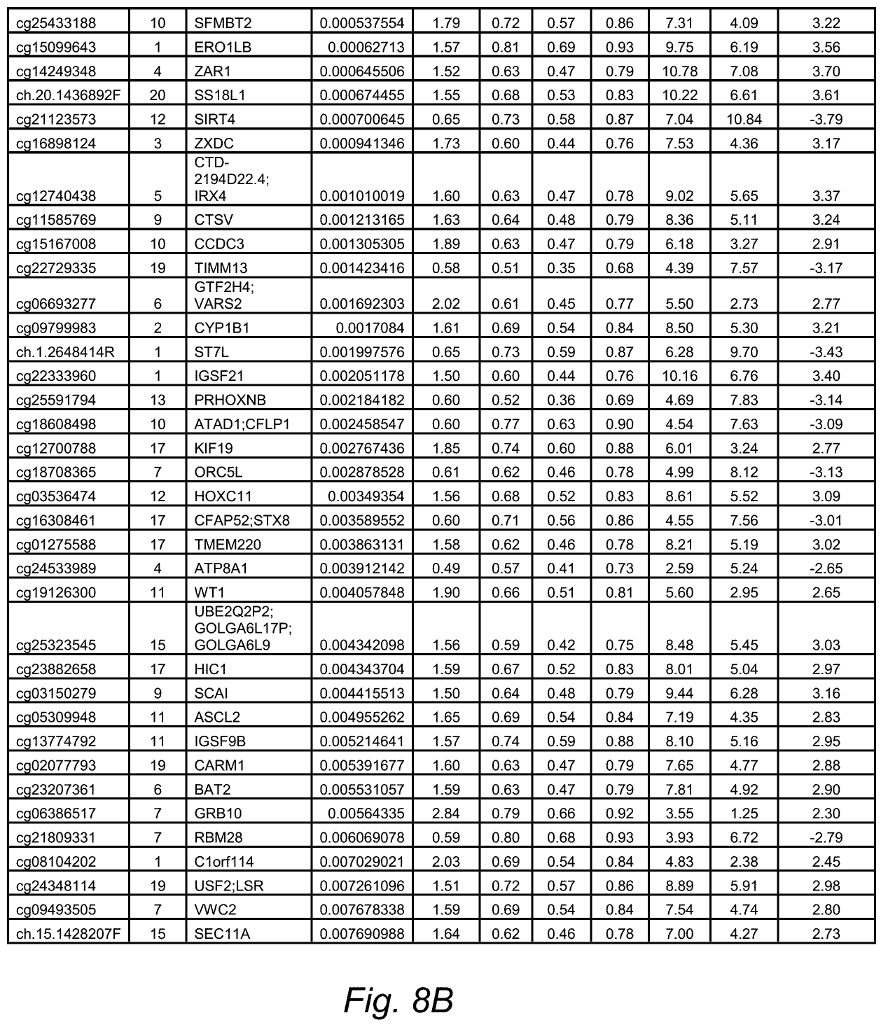

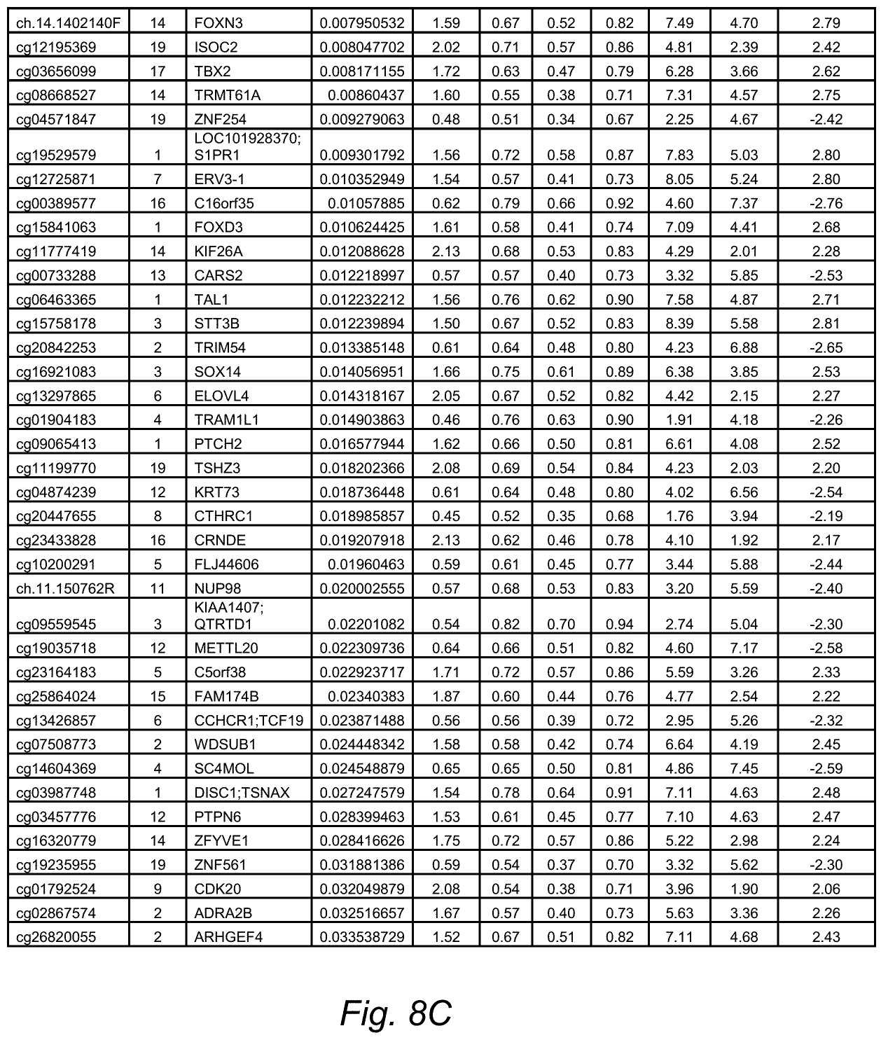

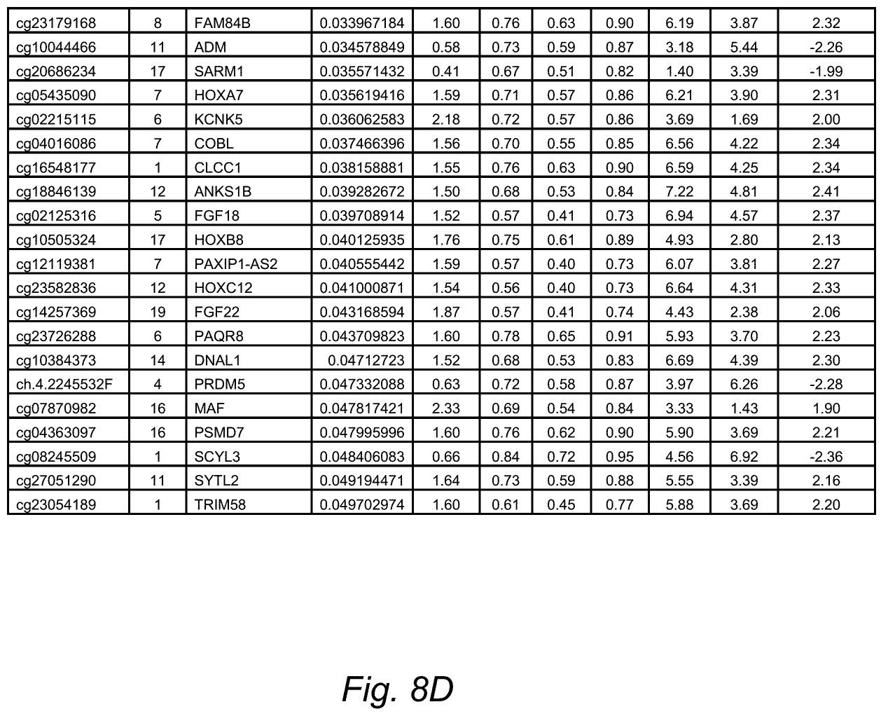

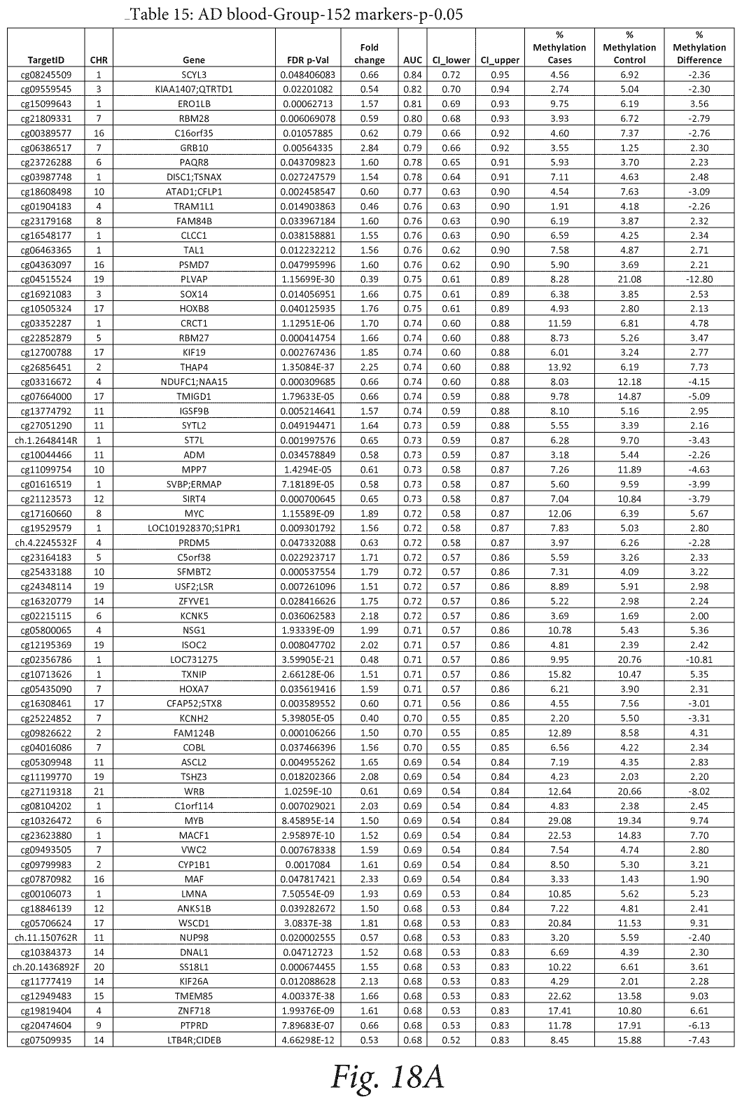

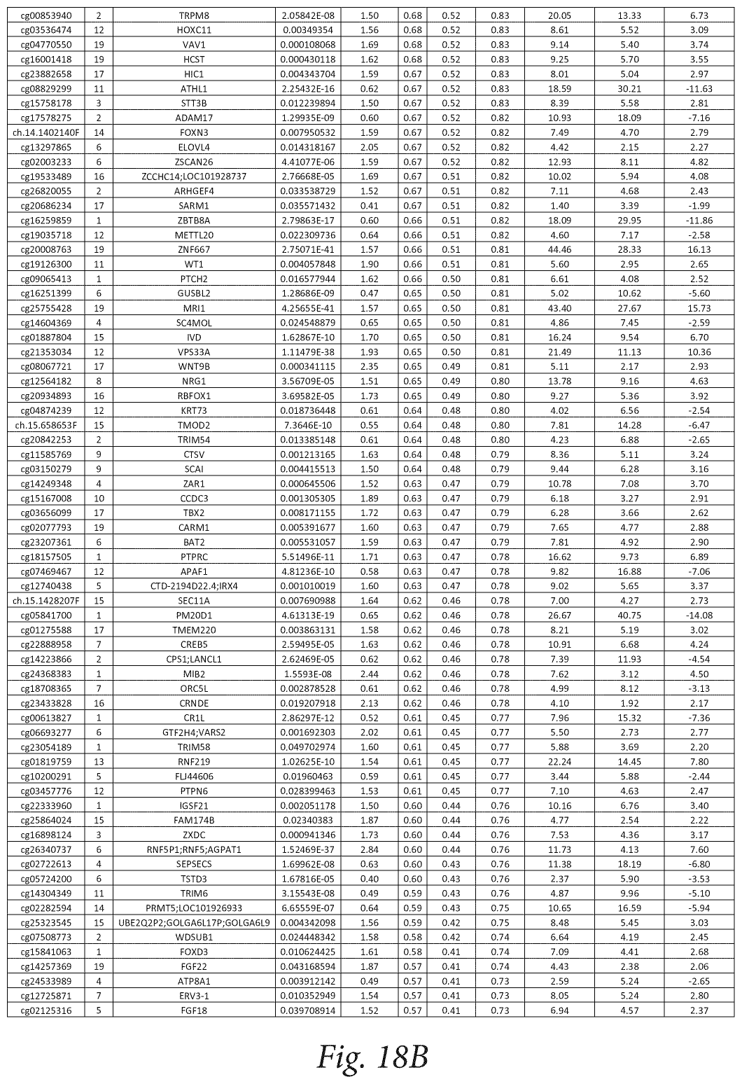

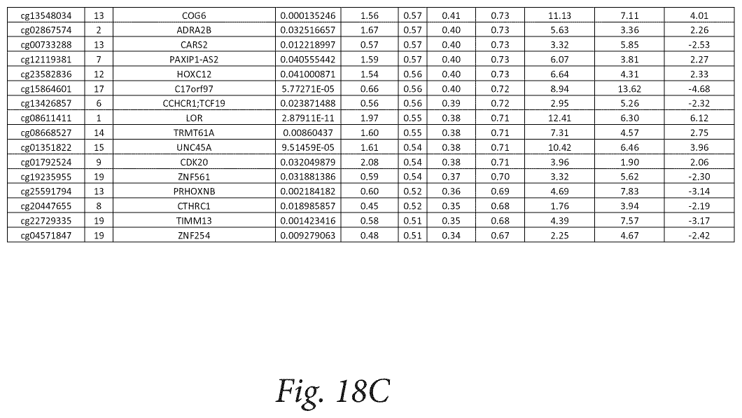

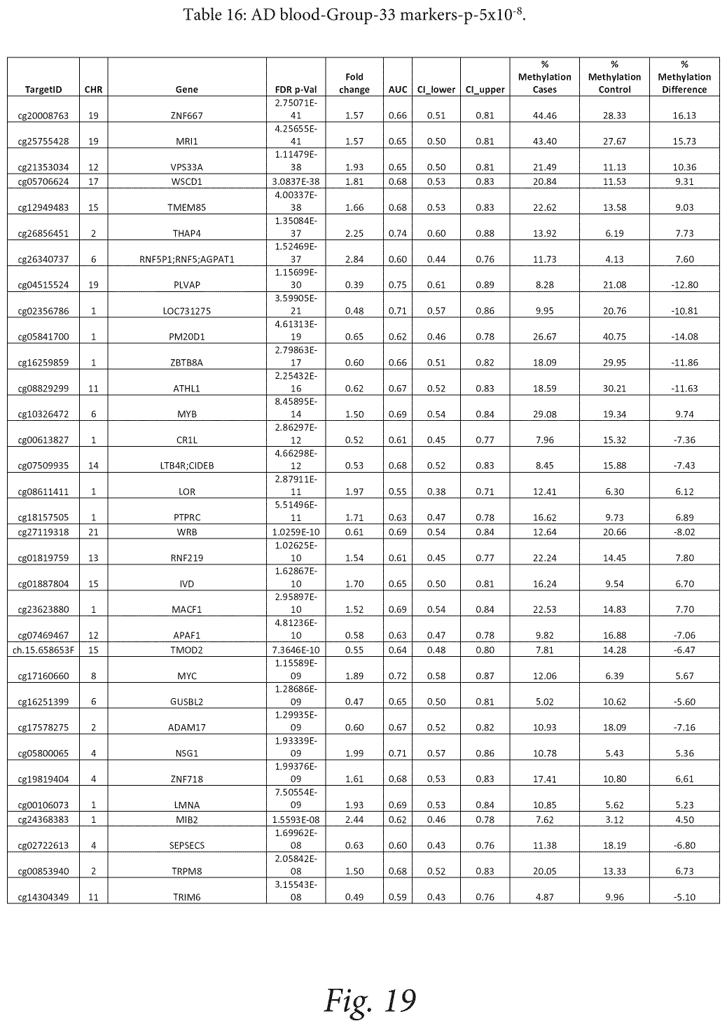

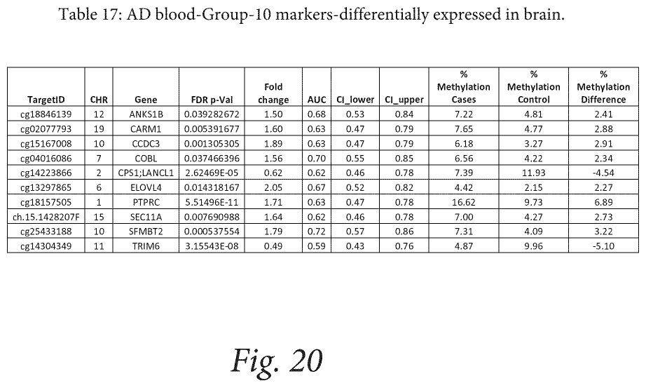

In still another variation, the plurality of Alzheimer indicators genes includes a plurality genes listed in FIGS. 4 to 20. In this regard, the plurality of Alzheimer indicators genes includes 1 percent to 80 percent of the genes listed in FIGS. 4 to 20. In a refinement, the plurality of Alzheimer indicators genes being evaluated includes 10 to 50 percent of the genes listed in FIGS. 4 to 20. In still other refinements, the plurality of Alzheimer indicators genes being evaluated includes greater than about, in increasing order of preference, 1%, 2%, 5%, 10%, 20%, 30% or 40% percent of the genes listed in FIGS. 4 to 20 and less than in increasing order of preference 100%, 90%, 80%, 70%, 60%, or 50% of the genes listed in FIG. 4-20.

In another embodiment, a method for diagnosing Alzheimer's Disease or determining susceptibility to Alzheimer's Disease is provided. The method includes steps of obtaining a blood sample from a target subject and performing cytosine methylation analysis of genes in leucocytes in the blood sample. A trained neural network is applied to determine if the target subject is at risk for or has Alzheimer's disease. Characteristically, the trained neural network is trained from genome-wide methylation test sets that include a first group of testing subjects having Alzheimer's disease and a second group of test subjects not having Alzheimer's disease diagnosed my current antemortem tests including clinical history and physical exam, psychological testing and imaging techniques including MM. Post mortem confirmation of the diagnosis can further be achieved by pathological examination of the brain specimens to identify the characteristic histological changes that are the gold standard for confirmation of AD. In a refinement, the data is randomly split as an 80% training set and the 20% as the test set. Detail for training and applying the neural network were performed analogously to the methods set forth in Bahado-Singh, R. O.; Vishweswaraiah, S.; Aydas, B.; Mishra, N. K.; Guda, C.; Radhakrishna, U. Deep Learning/Artificial Intelligence and Blood-Based DNA Epigenomic Prediction of Cerebral Palsy. Int. J. Mol. Sci. 2019, 20, 2075. https://doi.org/10.3390/ijms20092075; the entire disclosure of which is hereby incorporated by reference.

In a variation of the present embodiment, the method further includes a step of treating the target subject for Alzheimer's Disease is the target subject is identified as being at risk. In a refinement, the target subject is treated in clinical trial for Alzheimer's Disease if the target subject is identified as being at risk in a clinical trial. Early and accurate diagnosis is now regarded as critical for interventions for mitigating the disease, prolonging productive years and the identification of appropriate subjects for early intervention pharmacological trials.

In a variation, gene methylation analysis is performed genome-wide. In another variation, the gene methylation analysis is restricted to a plurality of previously identified Alzheimer indicator genes (or at least evaluation of the subject only relied on this plurality of previously identified Alzheimer indicator genes). More specifically the above refers to genes that have been reported to be differently expressed in the brains of patients who died of AD. In a refinement, the target subject is identified as having or being at risk for or has Alzheimer's Disease if the amount of methylation for one or more genes in the plurality of previously identified Alzheimer indicator genes differs from the amount of methylation measured in control subjects not having Alzheimer's Disease by a predetermined amount. In a refinement, the predetermined amount is at least a 30 percent difference in the amount of methylation as compared to control subjects (for corresponding genes between target subject and controls). The percent difference is (|control−target subject|/control)*100%). In other refinements, the predetermined amount is at least, in increasing order of preference, 30 percent, 50 percent, 100 percent or 200 percent difference in the amount of methylation as compared to control subjects (for corresponding genes between target subject and controls). Methylation levels are generally expressed as (beta) β-values. As per Illumina Corporation which manufactures the assay probes used, the β-value is defined as an estimate of the methylation level using the ratio of fluorescent intensities between fluoroscopic probes binding to methylated and unmethylated cytosine loci. β-value=Methylated allele intensity (M)/(Unmethylated allele intensity (U)+Methylated allele intensity (M). Thus for each cytosine locus, the average β-value is calculated for the AD group and also for the control group. The absolute percentage difference in methylation levels-increased (hypermethylated) or decreased (hypomethylation) can be determined. Conversely the fold change in methylation level in AD cases relative to controls e.g. >1.5 fold or >2.0 fold can be determined.

As set forth above, in some variations of the present embodiment, at least one gene in the plurality of Alzheimer indicators genes are hypomethylated in target subjects having or at risk for Alzheimer's Disease as compared to control subjects. Examples of such hypomethylated genes (more precisely target cytosine locus in the particular gene) include, but are not limited to PLVAP, KCNH2, TSTD3, SARM1, CTHRC1, TRAM1L1, GUSBL2, LOC731275, ZNF254 and TRIM6. In this regard, it should be appreciated that a target subject can be evaluated for one or any combination of these genes.

As set forth above, in some variations of the present embodiment, at least one gene (more precisely target cytosine locus in the particular gene) in the plurality of Alzheimer indicators genes are hypermethylated in target subjects having or at risk for Alzheimer's Disease as compared to control subjects. Examples of such hypermethylated genes include, but are not limited to RNF5P1, RNF5, AGPAT1, GRB10, MIB2, WNT9B, MAF, THAP4, KCNK5 and KIF26A. In this regard, it should be appreciated that a target subject can be evaluated for one or any combination of these genes.

As set forth above, in another variation of the present embodiment, at least one gene in the plurality of Alzheimer indicators genes are hypermethylated in target subjects having or at risk for Alzheimer's Disease as compared to control subjects. Examples of such hypermethylated genes (more precisely target cytosine locus in the particular gene) include, but are not limited to RNF5P1, RNF5, AGPAT1, GRB10, MIB2, WNT9B, MAF, THAP4, KCNK5 and KIF26A. In this regard, it should be appreciated that a target subject can be evaluated for one or any combination of these genes.

As set forth above, in still another variation of the present embodiment, the plurality of Alzheimer indicators genes includes a plurality genes listed in FIGS. 4 to 20. In this regard, the plurality of Alzheimer indicators genes includes 1 percent to 80 percent of the genes listed in FIGS. 4 to 20. In a refinement, the plurality of Alzheimer indicators genes being evaluated includes 10 to 50 percent of the genes listed in FIGS. 4 to 20. In still other refinements, the plurality of Alzheimer indicators genes being evaluated includes greater than about, in increasing order of preference, 1%, 2%, 5%, 10%, 20%, 30% or 40% percent of the genes listed in FIGS. 4 to 20 and less than in increasing order of preference 100%, 90%, 80%, 70%, 60%, or 50% of the genes listed in FIG. 4-20.

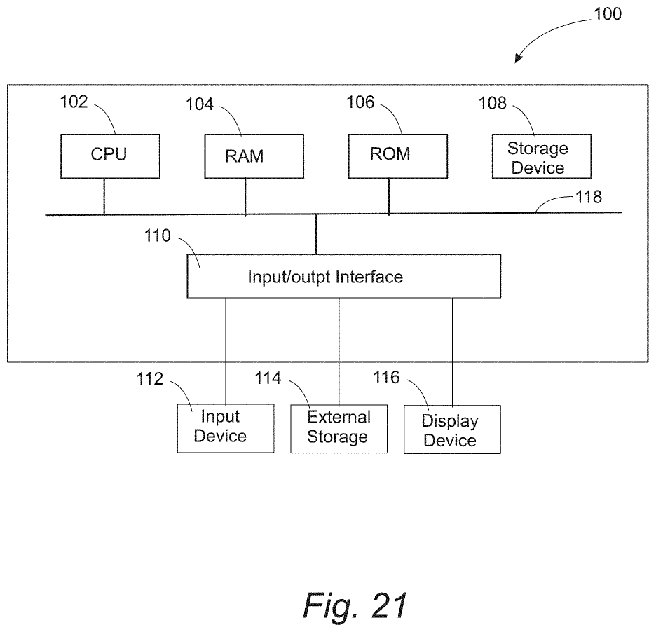

The methods set forth for diagnosing Alzheimer's Disease or determining susceptibility to Alzheimer's Disease and related training can be implemented by specialized hardware design for that purpose. More commonly, these steps can be implemented by a computer program executing on a computing device. FIG. 21 provides a block diagram of a computing system that can be used to implement the methods. Computing system 100 includes a processing unit 102 that executes the computer-readable instructions for diagnosing Alzheimer's Disease or determining susceptibility to Alzheimer's Disease and related training. Processing unit 102 can include one or more central processing units (CPU) or microprocessing units (MPU). Computer system 100 also includes RAM 104 or ROM 106 that can have instructions encoded thereon for the methods for diagnosing Alzheimer's Disease or determining susceptibility to Alzheimer's Disease and related training. Computer system 100 can also include a secondary storage device 108, such as a hard drive. Input/output interface 110 allows interaction of computing device 100 with an input device 112 such as a keyboard and mouse, external storage 114 (e.g., DVDs and CDROMs), and a display device 116 (e.g., a monitor). Processing unit 102, the RAM 104, the ROM 106, the secondary storage device 108, and input/output interface 110 are in electrical communication with (e.g., connected to) bus 118. During operation, computer system 100 reads computer-executable instructions (e.g., one or more programs) recorded on a non-transitory computer-readable storage medium which can be secondary storage device 108 and or external storage 114. Processing unit 102 executes these reads computer-executable instructions for diagnosing Alzheimer's Disease or determining susceptibility to Alzheimer's Disease and related training set forth above. Specific examples of non-transitory computer-readable storage medium for which executable instructions for diagnosing Alzheimer's Disease or determining susceptibility to Alzheimer's Disease and related training are encoded onto include but are not limited to, a hard disk, RAM, ROM, an optical disk (e.g., compact disc, DVD), or Blu-ray Disc (BD)™), a flash memory device, a memory card, and the like.

Mild cognitive impairment (MCI), is associated with a slight but observable decline in in cognition. For example memory and executive or thinking functions can be noticeably impaired. Individuals with MCI are at increased risk for the development of AD. A significant percentage of subjects with amnestic MCI will progress to AD (Kelley B J, Petersen R C. Alzheimer's disease and mild cognitive impairment Neurologic Clinics 2007; 25:577-609) although some can regress away from AD. MCI is therefore often a transitional condition leading to AD. Therefore, the biomarkers, methods and AI systems set forth above are relevant to and can be used for MCI detection and the prediction of MCI cases that will develop AD.

The following examples illustrate the various embodiments of the present invention. Those skilled in the art will recognize many variations that are within the spirit of the present invention and scope of the claims.

### SUMMARY

The utility of leucocyte DNA epigenomic biomarkers to detect AD and to elucidate its molecular pathogeneses is evaluated. Genome-wide DNA methylation analysis was performed using the Infinium MethylationEPIC array (Illumina). A cohort of 24 late-onset AD subjects and 24 unaffected control subjects were utilized. Methylation levels (β-value) at individual CpG loci were evaluated for AD detection. Significant differential methylation of individual CpG loci in AD versus controls was defined as false discovery rate (FDR) p-value<0.05. Given the large amounts of genomic data generated, we used six different Artificial Intelligence (AI) approaches including Deep Learning (DL) for AD detection. Detection performance was determined based on area under the receiver operating characteristics curve (AUC) and 95% CI, sensitivity and specificity. Ingenuity Pathway Analysis was used to identify the molecular and disease pathways that were found to be significantly epigenetically dysregulated in association with AD.

There were 152 differentially-methylated CpG loci (FDR p-value<0.05) associated with 171 separate genes in AD versus controls. The different AI techniques achieved high diagnostic accuracy for AD detection. At peak performance, each had an AUC>0.95. DL had an AUC (95% CI)=1.0 (0.8-1.0), with a sensitivities of 97.5-98% and specificities of 98-100% using CpG markers alone. Conventional clinical predictors e.g. psychological testing, age and gender did not further improve predictive accuracy. Epigenetically dysregulated molecular pathways (and genes) include some previously thought to be involved in AD development. These included genes involved in abnormal morphology of the cerebral cortex (CR1L, CTSV), gliosis (S1PR1), hydrocephalus (MYB, CYP1B1), inflammatory response (LTB4R). Cardiovascular disorder i.e. ventricular hypertrophy and dilation (CTSV, PRMT5) were also found to be associated with AD. The latter is particularly interesting given the increasing recognition of the role of cardiovascular disorders as a major risk factor for dementia development. In conclusion, our preliminary data suggest a novel, non-invasive approach, using AI and blood leucocyte epigenomics, for the accurate prediction and interrogating the mechanism of AD. Finally, our data support a significant role of epigenetic modification in the pathogenesis of AD.

Materials and Methods

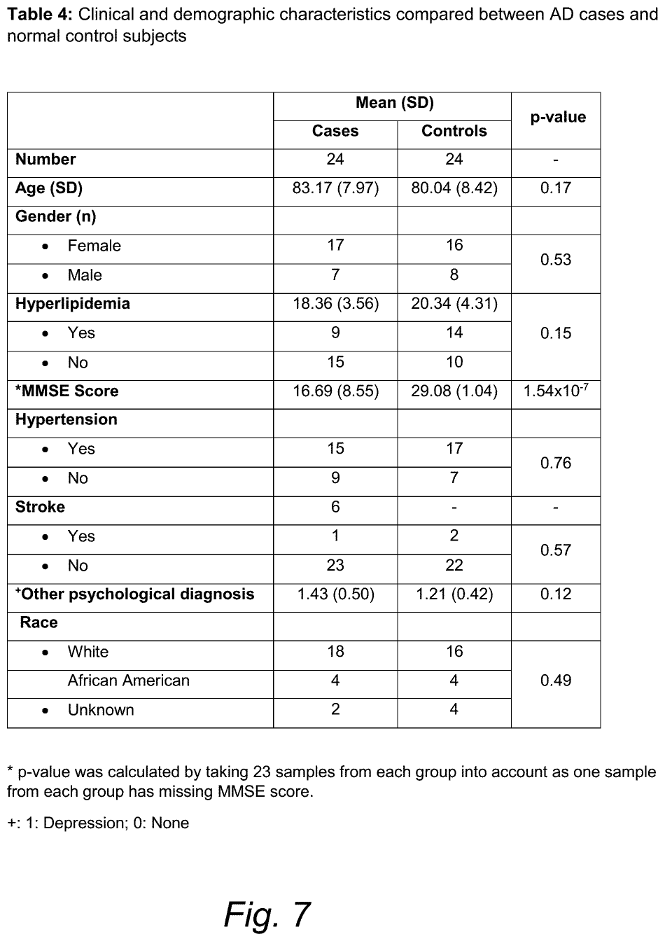

IRB approval was provided by the Institutional Review Board from William Beaumont Hospital, Royal Oak Mich., USA to perform this study. Written consent was obtained to perform the study. Subjects with a known or suspected genetic syndrome were excluded from participation. Demographic and clinical data were abstracted from the medical records (FIG. 7, Table 4) and compared between AD and control groups. Genomic DNA (gDNA) was extracted from whole blood samples using Gentra Puregene Blood Kit (Qiagen) according to the manufacturer's protocol. About 500 ng of genomic DNA were extracted from each of the 48 samples were bisulfite converted using the EZ DNA Methylation-Direct Kit (Zymo Research, Orange, Calif.) per the manufacturer's protocol and processed according to Illumina protocols. Bisulfite conversion was obtained in a PCR cycling protocol (i.e. 16×(95° C. for 30 sec, 50° C. for 60 min) and then hold at 4° C.) that is recommended for methylation analysis on the Infinium HumanMethylation Epic array BeadChip (Illumina, San Diego, Calif., USA). The Institutional Review Board from Beaumont health, Royal Oak, Mich., USA approved the study.

Genome-wide methylation scan using the Infinium MethylationEPIC array BeadChips. The Infinium MethylationEPIC array for methylation (Illumina, Inc., California, USA) that contains >850,000 CpGs per sample in the enhancer regions, gene bodies, promoters and CpG islands at a single-nucleotide resolution were used in the present study. MethylationEPIC array processing and methylome profiling were done according to manufacturer's protocol. Control and case samples were randomized on the arrays to minimize batch effect. Fluorescently-stained BeadChips were imaged by the Illumina iScan. Data was analyzed with GenomeStudio Software (Illumina) for methylation analysis. Prior to detailed bioinformatic and statistical analysis, data preprocessing and quality control were performed including examination of the background signal intensity of negative controls, the methylated and unmethylated signals, and the ratio of the methylated and unmethylated signal intensities. The β-value, an estimate of the methylation level at each CpG locus was calculated as per manufacturer specifications and was equal to the intensity of the methylated allele divided by the sum of the intensity of the methylated plus unmethylated alleles.

Removal of confounding factors. To avoid potential confounding, probes associated with X and Y chromosomes and/or containing SNPs in the probe sequence (listing dbSNP entries near or within the probe sequence, i.e., within 10 bp of the CpG site) are excluded from further analysis (Chen et al. 2013; Liu et al. 2013b; Wilhelm-Benartzi et al. 2013). Probes targeting CpG loci associated with SNPs near or within the probe sequence may influence corresponding methylated probes (Daca-Roszak et al. 2015). The remaining CpG sites are then analyzed. Data were normalized using the Controls Normalization Method. To avoid batch effect, all samples were processed together.

Validation of methylation results. Pyrosequencing was performed to test whether our methylation findings were robust. We validated 20 CpGs by pyrosequencing and confirmed the top-ranking hits in whole blood DNA of our cohort samples. These analyses revealed similar methylation data as those calculated from the Illumina Infinium MethylationEPIC arrays for all 20 genes. Detailed methodology was published previously (Radhakrishna et al. 2016).

Statistical and Bioinformatic analysis. A DNA methylation B-value were assigned to each CpG site. Differential methylation was assessed by comparing the B-values for each discrete cytosine nucleotide at each CpG site between AD subjects and controls. The p-value for methylation differences between AD and control groups at each locus was calculated as previously described (Altorok et al. 2014). To identify the significantly differentially methylated cytosines a False Discovery Rate (FDR) P-value threshold <0.05 with a Benjamini-Hochberg correction for multiple testing was utilized. Further a >1.5-fold threshold change in methylation was used to define methylation changes that were more likely to be biologically significant. The predictive ability of individual CpG loci for AD prediction was represented by area under the Receiver Operating Characteristics (ROC) curve and 95% CI.

Principal component analysis (PCA): Principal component analysis is a dimensional reduction technique that starting out with a large number of explanatory variables reduces the data-set to a smallest number of variables that significantly accounts for the difference between the study and control groups. Partial least squares discriminant analysis (PLS-DA) rotates the principal components (explanatory variables) in the PCA analysis to identify the optimal combination of principal components for discriminating the two groups. We used these approaches to determine whether epigenetic markers significantly differentiated AD from control groups (Chong & Xia 2018). Permutation testing was performed to determine whether the observed separation of the AD and control groups in the PCA analysis was statistically significant. This approach is often used n the evaluation of omics data.

Artificial Intelligence Analysis: AI analysis was performed for the detection of AD based on the methylation levels of a combination of CpG loci in different genes. The methods utilized have been previously reported by our group (Bahado-Singh et al. 2019a).

Deep Learning (DL)/Artificial Intelligence (AI) analysis. The β-values were logged and scaled by auto scaling using its standard deviation. Quantile normalization was performed to minimize sample to sample difference.

Deep Learning (DL). The hidden first layer was activated by providing sample input to the first layer based on the best parameters. Remaining layers were processed by updating the weights and biases for each layer. We utilized back propagation to regulate the parameters for all hidden layers. Softmax classifier was used both to assign new labels to the samples. To tune the parameters of the DL model, h2o package of R module was used (Alakwaa et al. 2018; Candel et al. 2018).

Other machine learning algorithms. We compared the performance of DL to five other machine learning algorithms: Support vector machine (SVM), Generalized linear Model (GLM), Prediction Analysis for Microarrays (PAM), Random Forest (RF) and Linear Discriminant Analysis (LDA) were performed on the AD data for the purpose of classification and regression analysis (Alakwaa et al. 2018). The Caret package in R module was used to achieve optimal predictive performance and to tune the parameters in the models, (Kuhn 2008). The pROC package of R module was used to compute AUC for AD detection and to assess the overall model performances.

AD prediction based on Artificial Intelligence approaches. Statistically significant individual CpG markers for individual prediction of AD were identified. These were limited to loci with significant methylation differences between cases and controls (defined as Benjamini-Hochberg FDR p-value<0.05). The individual predictive accuracy of each of these loci were determined based on a significant AUC (95% CI), and sensitivity and specificity values. Further, prediction was repeated but limited to the CpG loci with stringently defined methylation differences, AD versus controls, i.e. p-value<5×10⁻⁸. This threshold is recommended to ensure reproducibility and generalizability of findings in GWAS studies (Jannot et al. 2015). Analyses were performed using each of the six ML approaches.

Logistic Regression Analysis:

Conventional logistic regression analysis for AD prediction based on CpG methylation was also performed using the MetaboAnalyst v4.0 (Chong & Xia 2018) program for the purposes of comparison with the AI data. Cross validation analysis was done so that screening performance in both a test group and a subsequent validation group could be determined. Predictive accuracy was represented by AUC (95% CI), sensitivity and specificity for test and validation data sets.

Sum normalized, log transformed, and auto-scaled epigenetics data were further subjected to logistic regression analysis using the Biomarker function in MetaboAnalyst v4.0. To select the predictor variables used in the logistic regression Least Absolute Shrinkage and Selection Operator (LASSO) and stepwise variable selection were utilized for optimizing all the model components. The logistic regression models based on CpG loci subsets were developed with a 10-fold cross-validation. The area under the receiver operating characteristics curve (AUROC or AUC), sensitivity and specificity values were calculated for the assessment of model performance.

Gene ontology analysis and functional enrichment. Differentially methylated genes (FDR p-value≤0.01) were analyzed using the Ingenuity Pathway Analysis (IPA) software (Qiagen) to identify biological functions or interacting molecular networks. All CpGs without mapping IDs in IPA were excluded from analysis. Only genes for which Entrez identifiers are available were further analyzed. Over-represented canonical pathways, biological processes and molecular processes were determined.

Results

There were 24 late-onset AD subjects and 24 unaffected control in the study. Selected clinical and demographic characteristics were compared between the AD and control groups (FIG. 7, Table 4). There were no significant differences in age, gender, common cardiovascular diseases between groups. The MMSE (mini mental status exam) is a psychological test commonly administered to screen for AD. As expected the MMSE test score was significantly lower in the AD than in the control group (p-value<1.54×10⁻⁷). Comparison of the methylation profiles between AD and control subjects revealed 152 differentially-methylated CpG loci associated with 171 unique genes each with at least 1.5-fold change in methylation level with a false discovery rate (FDR) p<0.05. There were 33 CpG sites with stringent p-value thresholds i.e. p-value<5×10⁻⁸. A total of 17 separate intragenic CpG loci had moderate to good individual predictive accuracy (AUC≥0.75) for AD detection based on the methylation levels. Table 1 (FIG. 4) presents the top 25 CpG targets: CpG IDs, University of California Santa Cruz (UCSC) gene name, chromosome, FDR p-value, fold-change, AUC values, and percentage of methylation in both cases and controls along with the percentage methylation difference between cases and controls. The methylation fold change for AD compared to control is also provided. The additional CpG markers with significant methylation differences (FDR p-value<0.05) between AD and controls is presented in Table 5. Both hyper and hypo-methylation were observed among CpG loci in the AD cases. The top ten hypomethylated genes (more precisely predictive cytosine locus within the gene) were: PLVAP, KCNH2, TSTD3, SARM1, CTHRC1, TRAM1L1, GUSBL2, LOC731275, ZNF254 and TRIM6. Similarly, the top 10 hypermethylated genes are, RNF5P1, RNF5, AGPAT1, GRB10, MIB2, WNT9B, MAF, THAP4, KCNK5 and KIF26A.

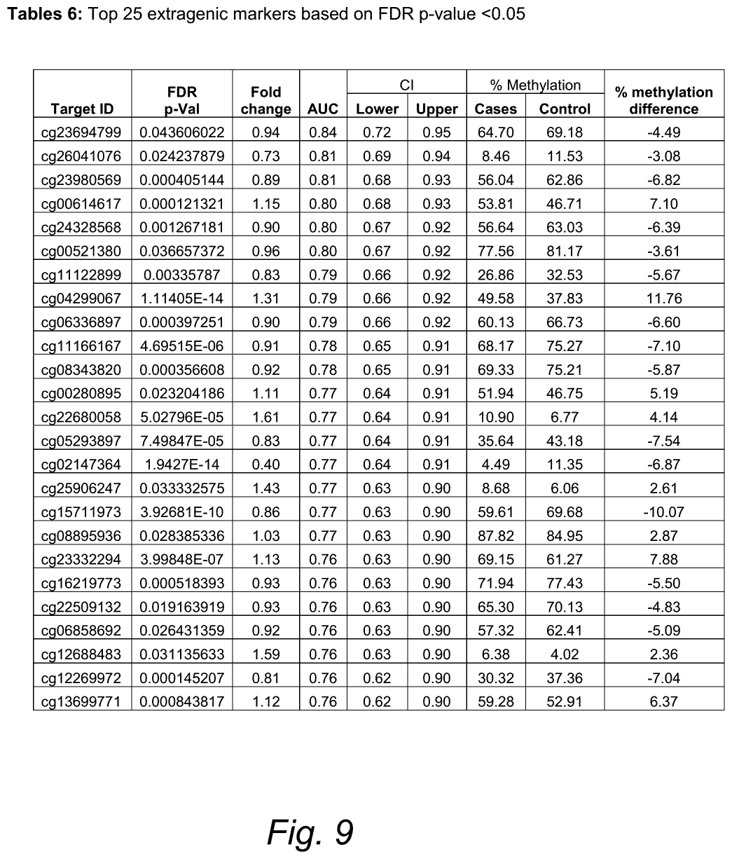

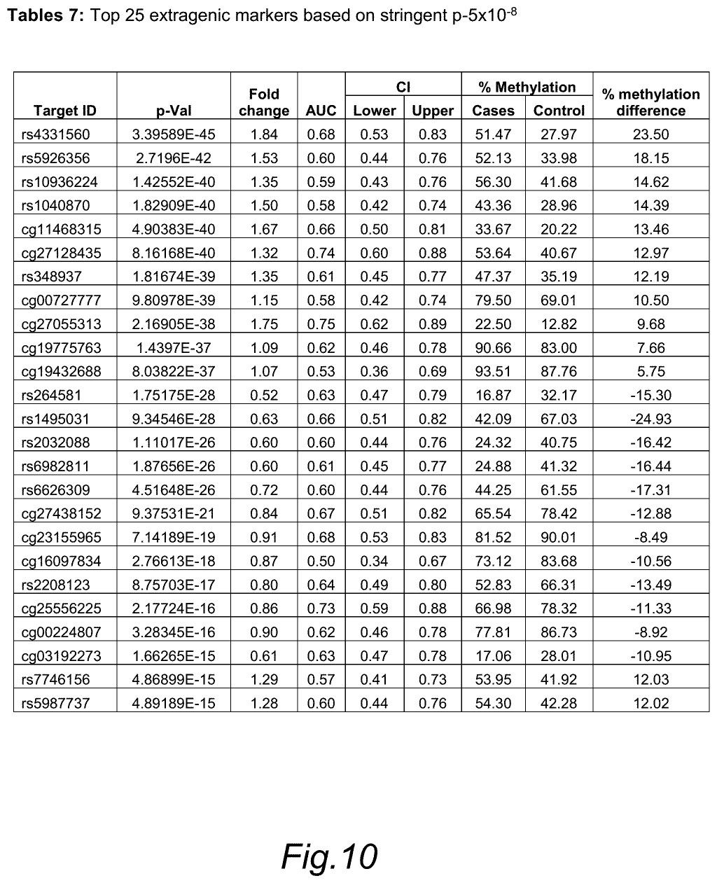

A prior report found significant differential methylation of extragenic sites in the genome in leucocytes in AD (Bollati et al. 2011) which correlated with the performance on the Mini-Mental State Examination (MMSE). Based on this we evaluated the methylation changes in extragenic CpG loci for AD prediction. Highly significant differences in CpG methylation was observed for multiple extragenic loci throughout the genome. This was observed when using different thresholds to define significance: i.e. FDR p-value<0.05 and also for stringent p-value thresholds, i.e. p-value<5×10⁻⁸. The top 25 extragenic markers for the different threshold values mentioned above are listed in Tables 6 and 10 (FIGS. 9 and 10).

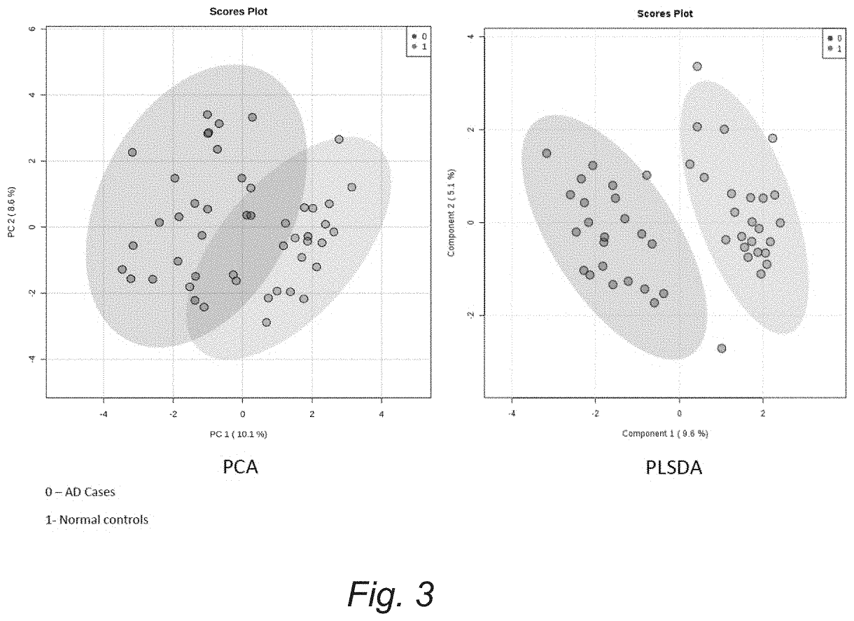

Principal Component Analysis (PCA) and Partial Least Square Discriminant Analyses confirmed significant segregation of AD cases from controls using (intragenic) CpG methylation markers (FIG. 3). Permutation analysis indicated that the separation observed between the two clusters was highly statistically significant (p=5e⁻⁰⁴) and not due to chance.

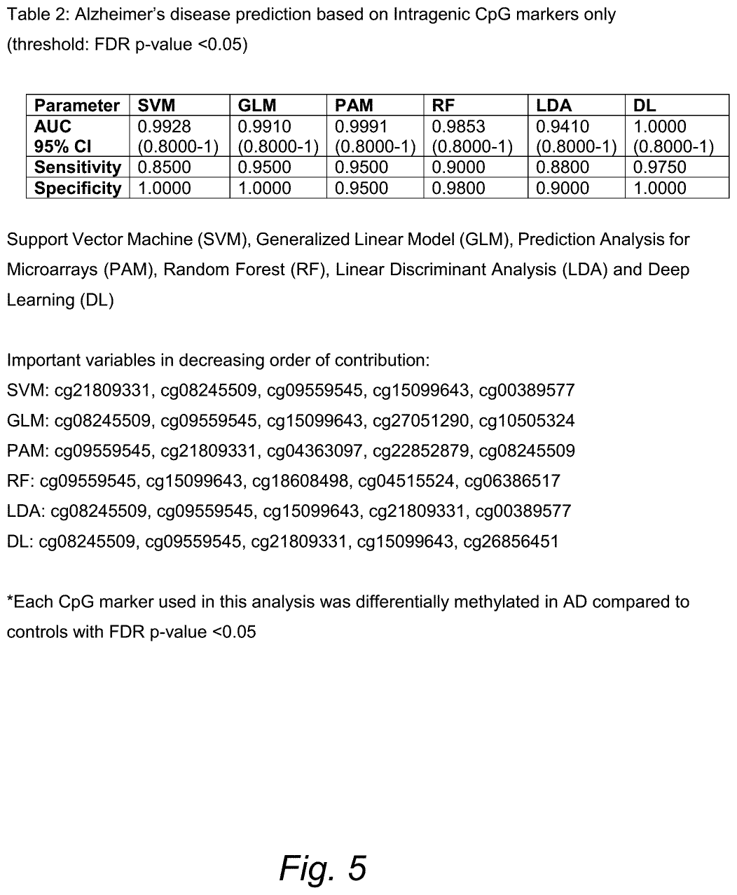

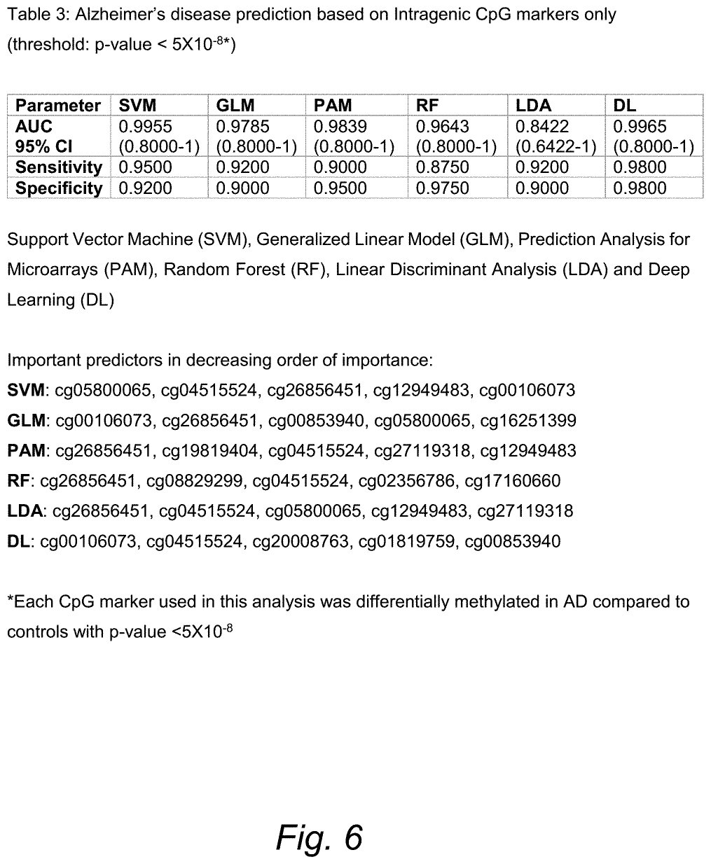

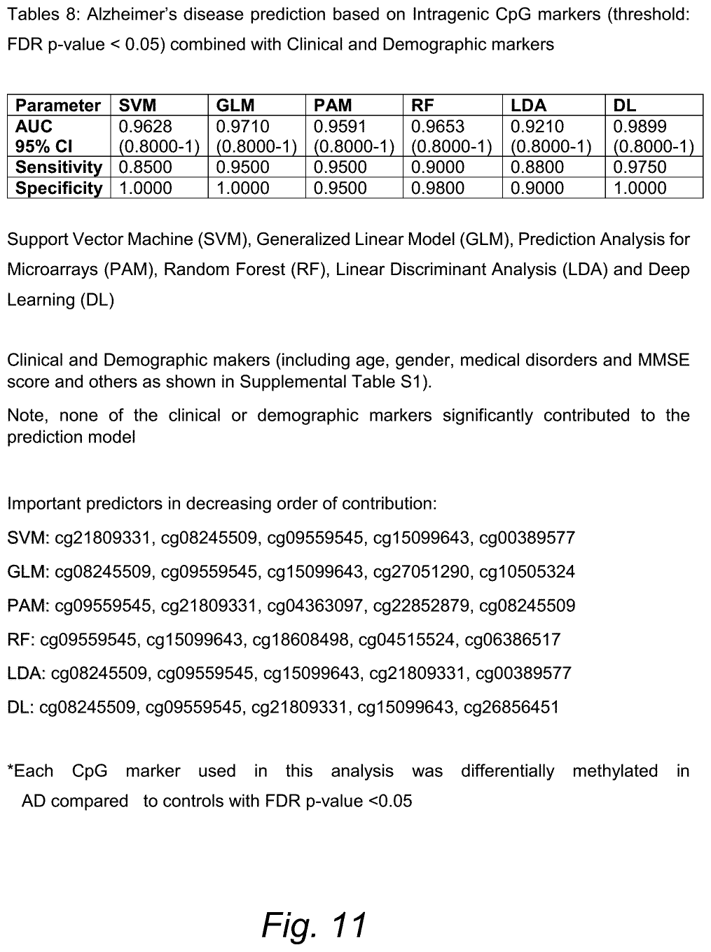

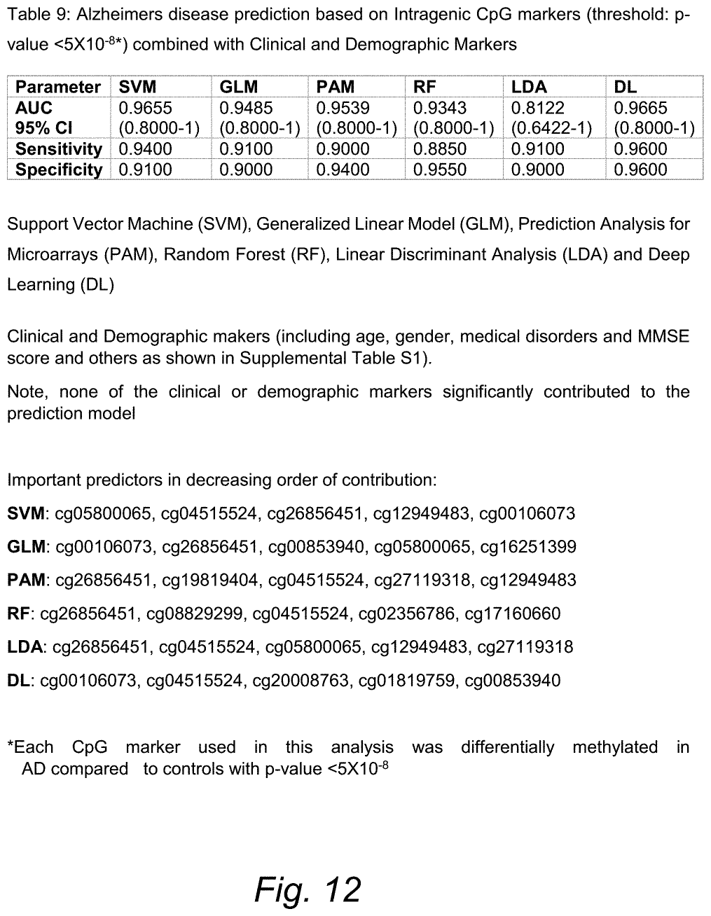

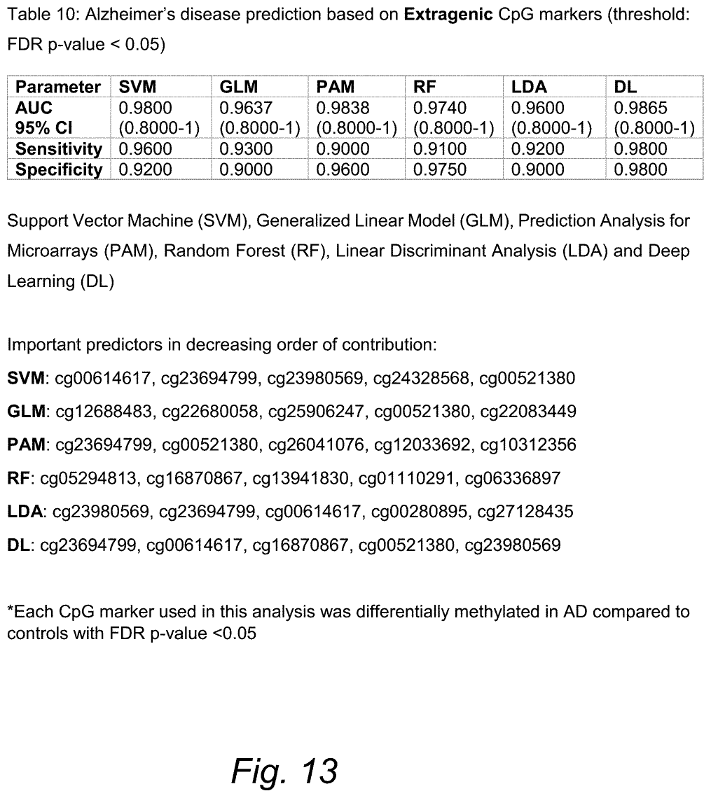

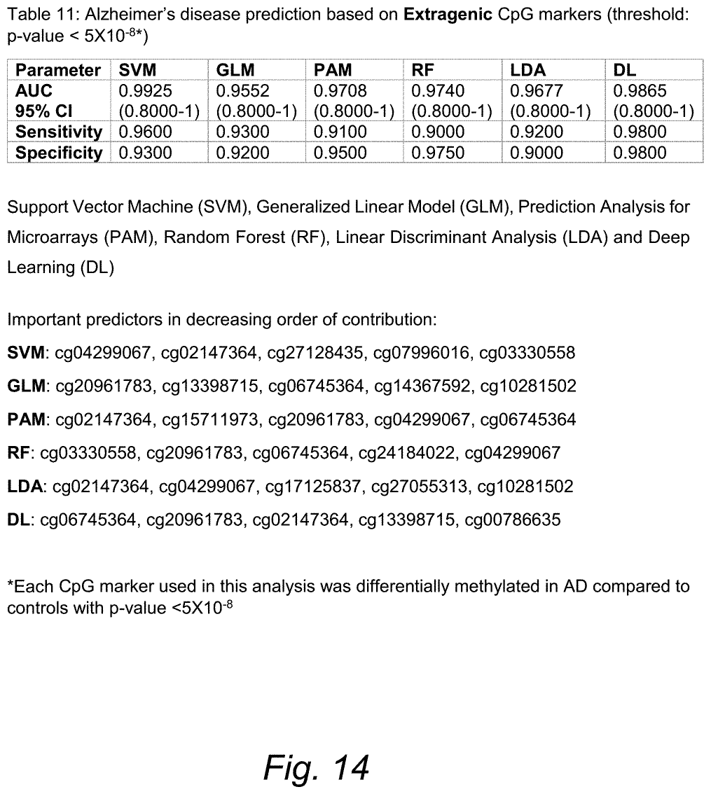

Machine Learning based only on methylation levels of CpG markers achieved highly accurate prediction of AD. This was true when individual CpG markers were limited to those with significance threshold set at: FDR p-value<0.05 (FIG. 5, Table 2) or using stringent criterion for significance p<5×10⁻⁸ (Jannot et al. 2015) (FIG. 6, Table 3). Almost all ML approaches had high predictive accuracy with an AUC≥0.95. In the case of Deep Learning, we observed an AUC=0.996-1.0 with sensitivities and specificities of 96-98% for AD prediction using the intragenic markers. The inclusion of a number of common clinical risk factors and predictors such as age, gender, medical disorders (such as hyperlipidemia and chronic hypertension) and MMSE scores did not significantly improve predictive accuracy over epigenomic makers alone (FIGS. 11 and 12, Tables 8 and 9). Further, we looked at the predictive performance of AI based on analysis of DNA methylation levels in extragenic CpG loci. High predictive accuracies were achieved with extragenic markers using the following significance thresholds: FDR p-value<0.05 (FIG. 13, Table 10) and p-value<5.0×10⁻⁸ (FIG. 14, Table 11).

Logistic Regression Analysis: Logistic regression analysis represents a more conventional approach for determining the predictive accuracy of biomarkers for disease prediction. Logistic regression models were also used for the prediction of AD and to confirm the robustness of epigenomic markers. The combination of cg04515524, cg00613827, cg02356786 and cg07509935; distinguished controls from ADs: AUC=0.856 (0.749˜0.963), sensitivity=0.917 (0.917˜1.000) and specificity=0.708 (0.526˜0.890) after 10-fold cross-validation. The logistic regression model built in this study is represented below:

logit(P)=log(P/(1−P))=−0.072−1.5 cg04515524−1.901 cg00613827−0.992 cg02356786−1.358 cg07509935

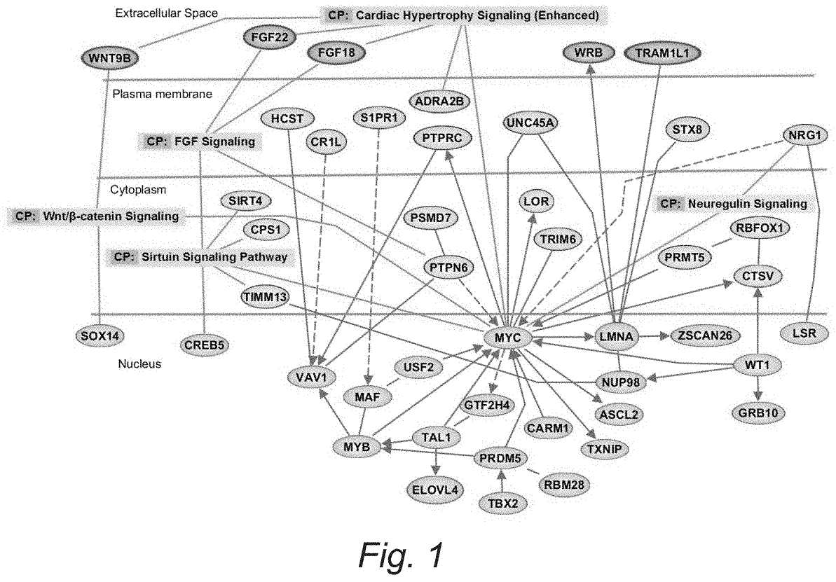

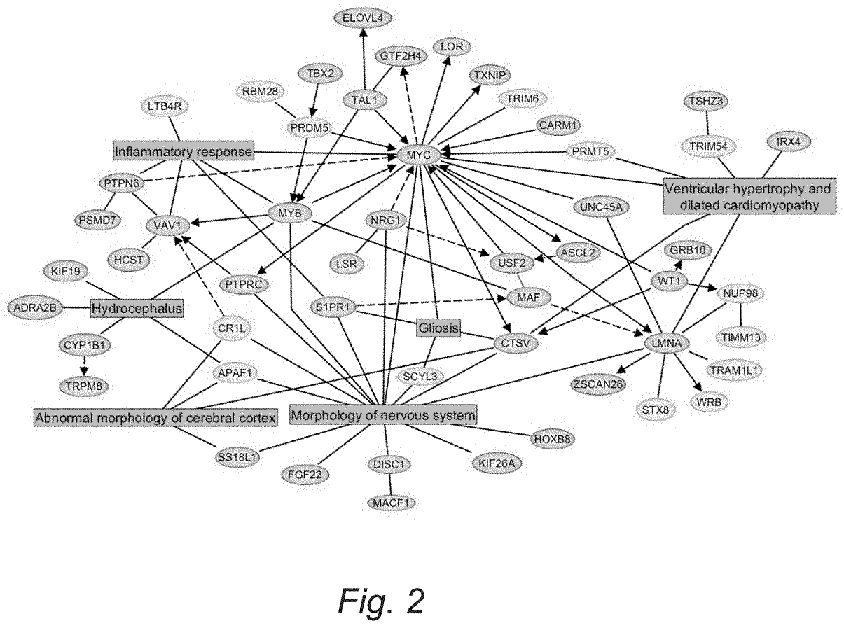

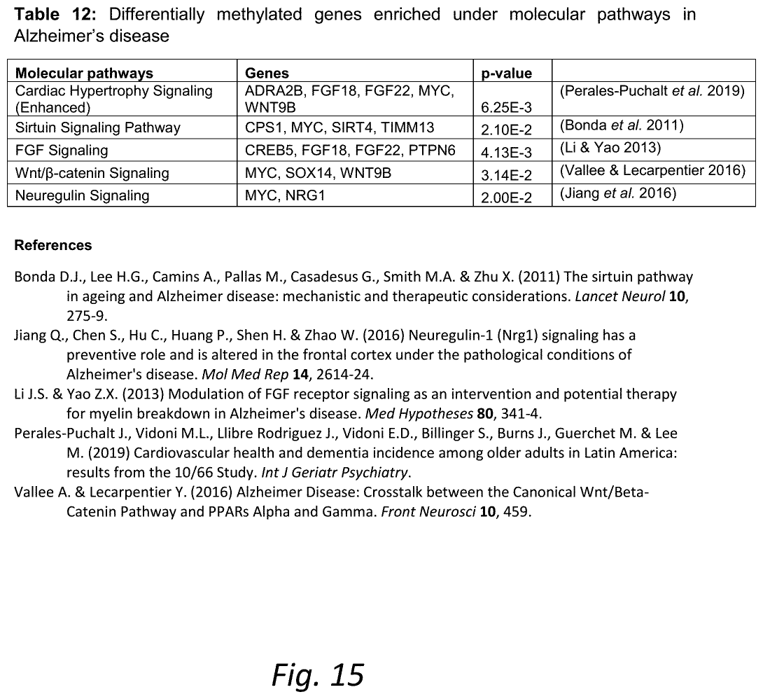

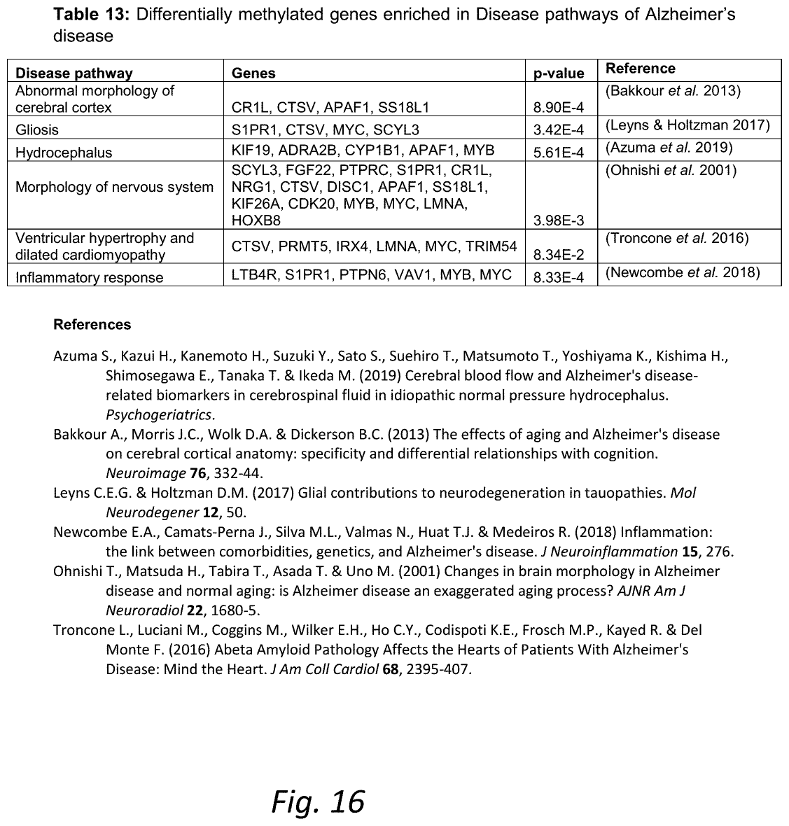

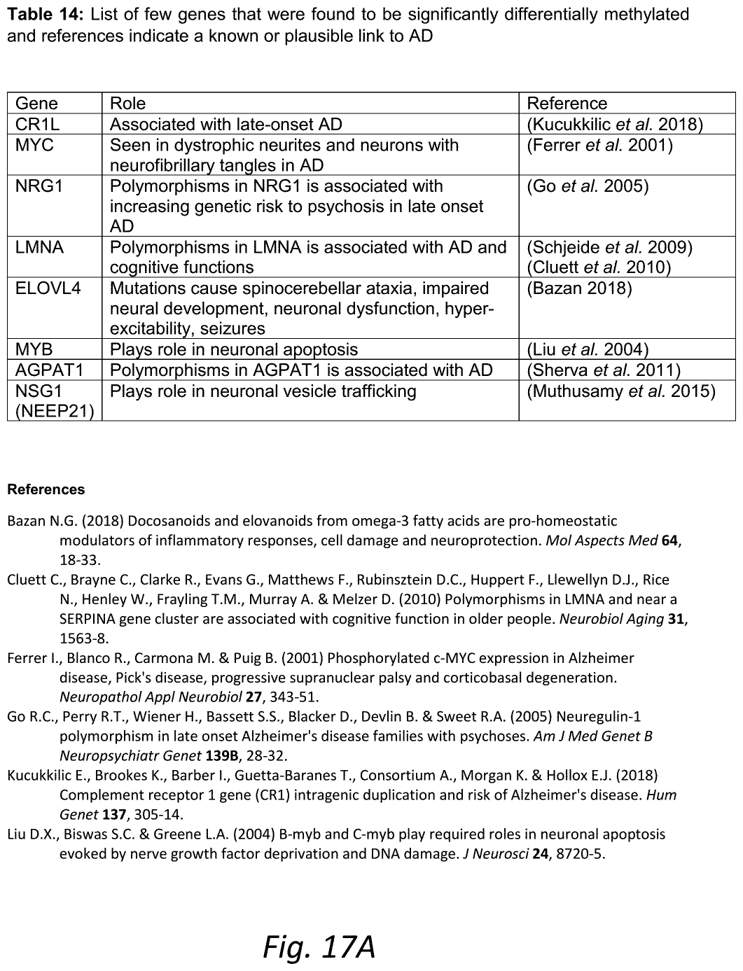

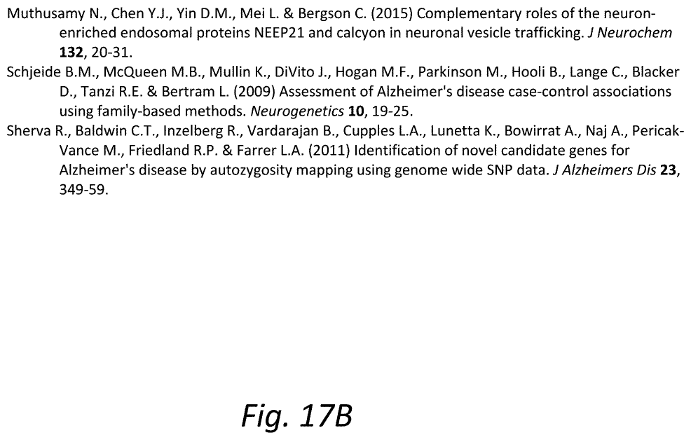

Network and Pathway Analyses Results. The network and pathway analysis identified five significantly enriched canonical pathways. The molecular pathways that were found to be statistically significantly overrepresented were Cardiac Hypertrophy Signaling (Enhanced), Sirtuin Signaling Pathway, FGF Signaling, Wnt/β-catenin Signaling and Neuregulin Signaling (FIG. 15, Table 12). The over-represented disease pathways were Abnormal morphology of cerebral cortex, Gliosis, Hydrocephalus, Morphology of nervous system, (cardiac) Ventricular hypertrophy, dilated cardiomyopathy and Inflammatory response (FIG. 16, Table 13). The related gene pathway (FIG. 1) and disease pathway (FIG. 2) are shown. Table 13 (FIG. 17) list some genes that were found to be significantly differentially methylated and references that indicate an established or plausible link to AD development.

### DISCUSSION

Dementia including AD represent the most significant crisis facing human health. The problem is expected to worsen with an anticipated explosion of the numbers of affected individuals in the future (Prince et al. 2015a). The direct health care costs are economically burdensome along with intangible costs at an estimated $550 billion (Hutubessy et al. 2003), is close to that for direct health care.

Despite the current absence of a specific therapy for AD, there is strong interest in the development of accurate and early biomarkers. The justification for biomarker development is compelling. Early detection of AD is needed to ensure early interventions that could potentially mitigate disease severity and help families to better prepare for the care of affected loved-ones. With a very active drug pipeline, early detection will be needed to identify appropriate candidates for drug trials. An important potential collateral benefit of biomarker development would be further elucidation of AD pathogenesis, thus facilitating the development of biology based targeted (precision) therapy. Finally, early detection and intervention to slow progression could minimize time spent with severe dementia and the preservation of cognitive function for as long as possible. This would be beneficial from the point of view of quality of life (Winblad et al. 2016) and health care costs. AD is a slowly developing disorder enhancing the feasibility of achieving these objectives.

As a consequence, the development of highly accurate biomarkers continues to be an urgent priority. Currently, a range of imaging markers continue to be deployed in clinical and research diagnosis and evaluation of AD. These include CT, MM, PET, imaging of the brain, and CSF amyloid levels. A systematic review of imaging biomarkers revealed that currently the most commonly utilized clinically available antemortem diagnostic tests have achieved moderate to good diagnostic accuracy (Cure et al. 2014). The expense and in some cases the invasive nature if these tests however preclude use except in the population at the highest risk. Psychological testing including the MMSE, the most widely used cognitive test, might not be readily available in many primary care settings where the majority of elderly patients receive clinical care and where early detection of AD would ideally occur. Further, the MMSE was found on meta-analysis to have only modest accuracy for ruling out dementia in a community or primary care settings (Mitchell 2009). Based on all these considerations, there is still a need for accurate screening tests in a low to moderate risk setting.

Consistent with the call for the integration of breakthrough technologies, systems biology, genomics, big data science and blood-based markers to advance precision medicine objectives in AD (Hampel et al. 2017), we combined AI analysis with blood epigenomic data for AD prediction. Using epigenetic markers alone we achieved highly accurate prediction of AD using different ML techniques. Almost all achieved an AUC≥0.95. In the case of Deep Learning, we found an AUC=0.996-1.0 with 97.5-98% sensitivity and specificities of 98-100%. Inclusion of a number of widely used clinical risk predictors such as age, gender, MMSE score and medical disorders including history of hypertension did not significantly improve predictive accuracy over epigenomic makers alone. Additionally, using AI we achieved high predictive accuracy using extragenic CpG loci for AD detection.

AI is superior to conventional statistical tools for analysis of big data generated by omics analysis (Bahado-Singh et al. 2019a; Mirza et al. 2019). We also looked however at the performance of conventional logistic regression approach. In a cross validated subset regression analysis demonstrated good predictive accuracy for AD using methylation markers, with an AUC (95% CI)=0.856 (0.749−0.963) with a regression approach. While AI performed better, this supports the robustness of the blood epigenomic markers for AD detection.

While not a requirement, a potential collateral benefit of the ideal biomarker, beyond prediction, is to elucidate disease pathogenesis. This provides biological plausibility indicating that indeed the affected genes are known or likely involved in brain function. Further, understanding the biological basis of disease is critical to the development of targeted treatment. We identified a number of genes that were significantly differentially methylated (Table 14, FIG. 17) that are known, suspected or could plausibly be linked to neuronal and brain disorders including Alzheimer's disease. In addition, gene networks were found to be epigenetically dysregulated (FIGS. 1 and 2) in AD providing significant insights into its pathogenesis. Overall, our data yielded evidence supporting the role of epigenetic modification in AD development. We briefly review selected genes and pathways found to be epigenetically dysregulated in AD.

Important Genes that were Epigenetically Modified in AD.

Briefly, we identified differential CpG methylation in several genes CR1L, MYC, NRG1, LMNA, ELOVL4, MYB, AGPAT1 and NSG1 previously believed to play a role in AD development. The associations include single nucleotide polymorphisms that increase AD risk while others are associated with functions such as the formation of neurofibrillary tangles, neuronal apoptosis and altered neuronal vesicle trafficking in that disorder (Table 14, FIG. 17) (Ferrer et al. 2001; Kucukkilic et al. 2018) (Liu et al. 2004; Go et al. 2005; Schjeide et al. 2009; Cluett et al. 2010; Sherva et al. 2011; Muthusamy et al. 2015; Bazan 2018). While very significant epigenetic changes were also identified in extragenic sites, we are currently unable to declare their contributory mechanisms to AD development. We will also now briefly discuss some of the disease mechanisms and also molecular pathways that were found to be associated with the leucocyte DNA methylation changes in AD.

Selected disease mechanisms enriched with epigenetically modified genes in AD: Abnormal morphology of cerebral cortex

AD appears to mainly affect the medial temporal cortex of the brain and both AD and aging affects the inferior parietal lobule and dorsolateral prefrontal cortex regions of the brain (Bakkour et al. 2013). The accumulation of a significant volume of neurofibrillary tangles in the neocortical region is a pre-requisite for AD development (Giannakopoulos et al. 1997). We found significant epigenetic changes in genes (CR1L, CTSV, APAF1 and SS18L1) responsible for cerebral cortical morphology.

Gliosis

Microglia are immune cells residing in brain which play important roles in neuroprotection and maintaining homeostasis. Proliferation and hypertrophy of these cells (‘gliosis’) occurs in response to central nervous system (CNS) damage. Gliosis can lead to neuroinflammation and induce tau pathology thus accelerating neurodegeneration. In the case of AD, amyloid-β plaque deposition aggravates gliosis (Leyns & Holtzman 2017). Our pathway analysis suggested a relationship between abnormal methylation and increased gliosis in AD. S1PR1 and MYC genes were hypermethylated in our study and enriched for gliosis. The S1PR1 gene is involved in CNS inflammation (Kim et al. 2018) and MYC gene in astrogliosis and inflammatory response (Takarada-Iemata et al. 2014).

Hydrocephalus

Normal-pressure hydrocephalus (NPH) is a clinical phenotype characterized by ventriculomegaly and disproportionally enlarged subarachnoid space with increased rates of cognitive impairment (Spina & Laws 2019). There is a greater than expected association between normal pressure hydrocephalus and late onset AD. In case series, up to 50% of NPH cases that underwent brain biopsy had neuropathological changes of AD in the cerebral cortex (Silverberg et al. 2003). It has thus been posited that reduced circulating CSF clearance in hydrocephalus could lead to increased accumulation of A-β. Conversely increased accumulation and deposition of Aβ in the meninges in AD reduces CSF outflow through these membranes, increasing ventricular volume, thus making NPH and AD mechanistically inter-related (Silverberg et al. 2003). Both MYB and CYP1B1 have been linked to hydrocephalus (Dragin et al. 2008; Malaterre et al. 2008). The two genes were found to be hypermethylated in our study.

Molecular Pathways Identified in AD

Cardiac Hypertrophy Signaling

Cardiovascular disease is strongly associated with cognition (Samieri et al. 2018). Left ventricular hypertrophy is reported to be an independent risk factor (Scuteri et al. 2009) for dementia. We identified several cardiovascular genes that were differentially methylated in AD. Polymorphisms of the ADRA2B gene are associated with cerebrovascular disorders (Kim et al. 2014). The FGF18 and FGF22 genes are known to play a role in heart development and physiological processes (Itoh et al. 2016) while the MYC gene is implicated in angiogenesis, cardiomyogenesis, apoptosis and oxidative stress response and plays major role in initiating and maintaining cardiac hypertrophy and contractility (Wolfram et al. 2011). These genes were found to be significantly differentially methylated in our study and further support a link between cardiovascular function and AD.

Wnt/β-Catenin Signaling

One potential mechanism by which cardiovascular disease and dementia are interlinked is through the Wnt/β-catenin signaling pathway. Wnt signaling is critical for multiple organ developmental processes including that of the heart. It is reactivated in many post-natal cardiac disorders (Foulquier et al. 2018). The activation of Wnt signaling has a neuroprotective effect while inhibition promotes neurodegeneration (Torres et al. 2019). Downregulated Wnt/β-catenin signaling has shown to be associated with AD (Vallee & Lecarpentier 2016). Wnt/β-catenin signaling genes: MYC, SOX14 and WNT9B were found to be hypermethylated in our study. Overall therefore, DNA methylation changes appeared based on our study to be biologically plausibly linked to AD.

While the numbers of subjects were modest, we demonstrated highly statistically significant methylation differences in AD cases and achieved accurate AD prediction based on CpG methylation markers. In addition, while expression studies were not performed in this particular analysis, in a number of CpG loci the difference in methylation levels in AD cases versus controls was greater than 5%-10%, a level generally associated with differences in gene expression (Leenen et al. 2016). A significant strength of our study is that it raises the prospect of non-invasive detection, investigation and monitoring of AD based on a blood test.

In summary, we have performed genome-wide methylation analysis in blood leucocytes and identified significant methylation changes in genes, gene networks and disease pathways that are known or suspected to play an important role in AD. Using AI techniques, highly accurate epigenomic prediction of AD was reported for the first time to the authors' knowledge in AD. This could potentially significantly advance the precision medicine objectives that have been outlined for AD (Hampel et al. 2017). Our work provides evidence in support for the view that epigenetic factors play a pivotal role in AD development.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

### REFERENCES

Alakwaa F. M., Chaudhary K. & Garmire L. X. (2018) Deep Learning Accurately Predicts Estrogen Receptor Status in Breast Cancer Metabolomics Data. J Proteome Res 17, 337-47.
Alpay Savasan Z., Yilmaz A., Ugur Z., Aydas B., Bahado-Singh R. O. & Graham S. F. (2019) Metabolomic Profiling of Cerebral Palsy Brain Tissue Reveals Novel Central Biomarkers and Biochemical Pathways Associated with the Disease: A Pilot Study. 9.
Altorok N., Tsou P. S., Coit P., Khanna D. & Sawalha A. H. (2014) Genome-wide DNA methylation analysis in dermal fibroblasts from patients with diffuse and limited systemic sclerosis reveals common and subset-specific DNA methylation aberrancies. Ann Rheum Dis.
Bahado-Singh R. O., Sonek J., McKenna D., Cool D., Aydas B., Turkoglu O., Bjorndahl T., Mandal R., Wishart D., Friedman P., Graham S. F. & Yilmaz A. (2018) Artificial Intelligence and amniotic fluid multiomics analysis: The prediction of perinatal outcome in asymptomatic short cervix. Ultrasound Obstet Gynecol. (in press).
Bahado-Singh R. O., Vishweswaraiah S., Aydas B., Mishra N. K., Guda C. & Radhakrishna U. (2019a) Deep Learning/Artificial Intelligence and Blood-Based DNA Epigenomic Prediction of Cerebral Palsy. Int J Mol Sci 20.
Bahado-Singh R. O., Yilmaz A., Bisgin H., Turkoglu O., Kumar P., Sherman E., Mrazik A., Odibo A. & Graham S. F. (2019b) Artificial intelligence and the analysis of multi-platform metabolomics data for the detection of intrauterine growth restriction. PLoS One 14, e0214121.
Bakkour A., Morris J. C., Wolk D. A. & Dickerson B. C. (2013) The effects of aging and Alzheimer's disease on cerebral cortical anatomy: specificity and differential relationships with cognition. Neuroimage 76, 332-44.
Bazan N. G. (2018) Docosanoids and elovanoids from omega-3 fatty acids are pro-homeostatic modulators of inflammatory responses, cell damage and neuroprotection. Mol Aspects Med 64, 18-33.
Berkel T. D. & Pandey S. C. (2017) Emerging Role of Epigenetic Mechanisms in Alcohol Addiction. Alcohol Clin Exp Res 41, 666-80.
Bollati V., Galimberti D., Pergoli L., Dalla Valle E., Barretta F., Cortini F., Scarpini E., Bertazzi P. A. & Baccarelli A. (2011) DNA methylation in repetitive elements and Alzheimer disease. Brain Behav Immun 25, 1078-83.
Candel A., Parmar V., LeDell E. & Arora A. (2018) Deep Learning with H2O.
Chen Y. A., Lemire M., Choufani S., Butcher D. T., Grafodatskaya D., Zanke B. W., Gallinger S., Hudson T. J. & Weksberg R. (2013) Discovery of cross-reactive probes and polymorphic CpGs in the Illumina Infinium HumanMethylation450 microarray. Epigenetics 8, 203-9.
Ching T., Himmelstein D. S., Beaulieu-Jones B. K., Kalinin A. A., Do B. T., Way G. P., Ferrero E., Agapow P. M., Zietz M., Hoffman M. M., Xie W., Rosen G. L., Lengerich B. J., Israeli J., Lanchantin J., Woloszynek S., Carpenter A. E., Shrikumar A., Xu J., Cofer E. M., Lavender C. A., Turaga S. C., Alexandari A. M., Lu Z., Harris D. J., DeCaprio D., Qi Y., Kundaje A., Peng Y., Wiley L. K., Segler M. H. S., Boca S. M., Swamidass S. J., Huang A., Gitter A. & Greene C. S. (2018) Opportunities and obstacles for deep learning in biology and medicine. J R Soc Interface 15.
Chong J. & Xia J. (2018) MetaboAnalystR: an R package for flexible and reproducible analysis of metabolomics data. Bioinformatics 34, 4313-4.
Cluett C., Brayne C., Clarke R., Evans G., Matthews F., Rubinsztein D. C., Huppert F., Llewellyn D. J., Rice N., Henley W., Frayling T. M., Murray A. & Melzer D. (2010) Polymorphisms in LMNA and near a SERPINA gene cluster are associated with cognitive function in older people. Neurobiol Aging 31, 1563-8.
Cure S., Abrams K., Belger M., Dell'agnello G. & Happich M. (2014) Systematic literature review and meta-analysis of diagnostic test accuracy in Alzheimer's disease and other dementia using autopsy as standard of truth. J Alzheimers Dis 42, 169-82.
Daca-Roszak P., Pfeifer A., Zebracka-Gala J., Rusinek D., Szybinska A., Jarzab B., Witt M. & Zietkiewicz E. (2015) Impact of SNPs on methylation readouts by Illumina Infinium HumanMethylation450 BeadChip Array: implications for comparative population studies. BMC Genomics 16, 1003.
Daviglus M. L., Bell C. C., Berrettini W., Bowen P. E., Connolly E. S., Jr., Cox N. J., Dunbar-Jacob J. M., Granieri E. C., Hunt G., McGarry K., Patel D., Potosky A. L., Sanders-Bush E., Silberberg D. & Trevisan M. (2010) NIH state-of-the-science conference statement: Preventing Alzheimer's disease and cognitive decline. NIH Consens State Sci Statements 27, 1-30.
De Jager P. L., Srivastava G., Lunnon K., Burgess J., Schalkwyk L. C., Yu L., Eaton M. L., Keenan B. T., Ernst J., McCabe C., Tang A., Raj T., Replogle J., Brodeur W., Gabriel S., Chai H. S., Younkin C., Younkin S. G., Zou F., Szyf M., Epstein C. B., Schneider J. A., Bernstein B. E., Meissner A., Ertekin-Taner N., Chibnik L. B., Kellis M., Mill J. & Bennett D. A. (2014) Alzheimer's disease: early alterations in brain DNA methylation at ANK1, BIN1, RHBDF2 and other loci. Nat Neurosci 17, 1156-63.
Dragin N., Shi Z., Madan R., Karp C. L., Sartor M. A., Chen C., Gonzalez F. J. & Nebert D. W. (2008) Phenotype of the Cyp1a1/1a2/1b1−/− triple-knockout mouse. Mol Pharmacol 73, 1844-56.
Ferrer I., Blanco R., Carmona M. & Puig B. (2001) Phosphorylated c-MYC expression in Alzheimer disease, Pick's disease, progressive supranuclear palsy and corticobasal degeneration. Neuropathol Appl Neurobiol 27, 343-51.
Foulquier S., Daskalopoulos E. P., Lluri G., Hermans K. C. M., Deb A. & Blankesteijn W. M. (2018) WNT Signaling in Cardiac and Vascular Disease. Pharmacol Rev 70, 68-141.
Giannakopoulos P., Hof P. R., Michel J. P., Guimon J. & Bouras C. (1997) Cerebral cortex pathology in aging and Alzheimer's disease: a quantitative survey of large hospital-based geriatric and psychiatric cohorts. Brain Res Brain Res Rev 25, 217-45.
Go R. C., Perry R. T., Wiener H., Bassett S. S., Blacker D., Devlin B. & Sweet R. A. (2005) Neuregulin-1 polymorphism in late onset Alzheimer's disease families with psychoses. Am J Med Genet B Neuropsychiatr Genet 139B, 28-32.
Grinan-Ferre C., Corpas R., Puigoriol-Illamola D., Palomera-Avalos V., Sanfeliu C. & Pallas M. (2018) Understanding Epigenetics in the Neurodegeneration of Alzheimer's Disease: SAMP8 Mouse Model. J Alzheimers Dis 62, 943-63.
Hampel H., O'Bryant S. E., Durrleman S., Younesi E., Rojkova K., Escott-Price V., Corvol J. C., Broich K., Dubois B., Lista S. & Alzheimer Precision Medicine I. (2017) A Precision Medicine Initiative for Alzheimer's disease: the road ahead to biomarker-guided integrative disease modeling. Climacteric 20, 107-18.
Hutubessy R., Chisholm D. & Edejer T. T. (2003) Generalized cost-effectiveness analysis for national-level priority-setting in the health sector. Cost Eff Resour Alloc 1, 8.
Itoh N., Ohta H., Nakayama Y. & Konishi M. (2016) Roles of FGF Signals in Heart Development, Health, and Disease. Front Cell Dev Biol 4, 110.
Jannot A. S., Ehret G. & Perneger T. (2015) P<5×10(−8) has emerged as a standard of statistical significance for genome-wide association studies. J Clin Epidemiol 68, 460-5.
Kim J. O., Jeon Y. J., Kim O. J., Oh S. H., Kim H. S., Shin B. S., Oh D., Kim E. J., Cho Y. K. & Kim N. K. (2014) Association between common genetic variants of alpha2A-, alpha2B- and alpha2C-adrenoceptors and the risk of silent brain infarction. Mol Med Rep 9, 2459-66.
Kim S., Bielawski J., Yang H., Kong Y., Zhou B. & Li J. (2018) Functional antagonism of sphingosine-1-phosphate receptor 1 prevents cuprizone-induced demyelination. Glia 66, 654-69.
Kucukkilic E., Brookes K., Barber I., Guetta-Baranes T., Consortium A., Morgan K. & Hollox E. J. (2018) Complement receptor 1 gene (CR1) intragenic duplication and risk of Alzheimer's disease. Hum Genet 137, 305-14.
Kuhn M. (2008) Building Predictive Models in R Using the caret Package. Journal of Statistical Software 28, 1-26.
Kusdra L., Rempel H., Yaffe K. & Pulliam L. (2000) Elevation of CD69+ monocyte/macrophages in patients with Alzheimer's disease. Immunobiology 202, 26-33.
Leenen F. A., Muller C. P. & Turner J. D. (2016) DNA methylation: conducting the orchestra from exposure to phenotype? Clin Epigenetics 8, 92.
Leyns C. E. G. & Holtzman D. M. (2017) Glial contributions to neurodegeneration in tauopathies. Mol Neurodegener 12, 50.
Li H., Guo Z., Guo Y., Li M., Yan H., Cheng J., Wang C. & Hong G. (2016) Common DNA methylation alterations of Alzheimer's disease and aging in peripheral whole blood. Oncotarget 7, 19089-98.
Liu C. C., Liu C. C., Kanekiyo T., Xu H. & Bu G. (2013a) Apolipoprotein E and Alzheimer disease: risk, mechanisms and therapy. Nat Rev Neurol 9, 106-18.
Liu D. X., Biswas S. C. & Greene L. A. (2004) B-myb and C-myb play required roles in neuronal apoptosis evoked by nerve growth factor deprivation and DNA damage. J Neurosci 24, 8720-5.
Liu J Chen J Ehrlich S Walton E White T Perrone-Bizzozero N Bustillo J., Turner J. A. & Calhoun V. D. (2014) Methylation patterns in whole blood correlate with symptoms in schizophrenia patients. Schizophr Bull 40, 769-76.
Liu Y., Aryee M. J., Padyukov L., Fallin M. D., Hesselberg E., Runarsson A., Reinius L., Acevedo N., Taub M., Ronninger M., Shchetynsky K., Scheynius A., Kere J., Alfredsson L., Klareskog L., Ekstrom T. J. & Feinberg A. P. (2013b) Epigenome-wide association data implicate DNA methylation as an intermediary of genetic risk in rheumatoid arthritis. Nat Biotechnol 31, 142-7.
Malaterre J., Mantamadiotis T., Dworkin S., Lightowler S., Yang Q., Ransome M. I., Turnley A. M., Nichols N. R., Emambokus N. R., Frampton J. & Ramsay R. G. (2008) c-Myb is required for neural progenitor cell proliferation and maintenance of the neural stem cell niche in adult brain. Stem Cells 26, 173-81.
Mamoshina P., Vieira A., Putin E. & Zhavoronkov A. (2016) Applications of Deep Learning in Biomedicine. Mol Pharm 13, 1445-54.
Mirza B., Wang W., Wang J., Choi H., Chung N. C. & Ping P. (2019) Machine Learning and Integrative Analysis of Biomedical Big Data. Genes (Basel) 10.
Mitchell A. J. (2009) A meta-analysis of the accuracy of the mini-mental state examination in the detection of dementia and mild cognitive impairment. J Psychiatr Res 43, 411-31.
Muthusamy N., Chen Y. J., Yin D. M., Mei L. & Bergson C. (2015) Complementary roles of the neuron-enriched endosomal proteins NEEP21 and calcyon in neuronal vesicle trafficking. J Neurochem 132, 20-31.
Prince M., Wilmo A., Guerchet M., Ali G., Wu Y. & Prina M. (2015a) World Alzheimer Report 2015: The global impact of dementia. An analysis of prevalence, incidence, costs and trends. London: Alzheimer's Disease International 2015. Alzheimer's Disease International. The global voice of dementia.
Prince M. J., Wimo A., Guerchet M. M., Ali G. C., Wu Y.-T. & Prina M. (2015b) World Alzheimer Report 2015—The Global Impact of Dementia. Alzheimer's Disease International, London.
Radhakrishna U., Albayrak S., Alpay-Savasan Z., Zeb A., Turkoglu O., Sobolewski P. & Bahado-Singh R. O. (2016) Genome-Wide DNA Methylation Analysis and Epigenetic Variations Associated with Congenital Aortic Valve Stenosis (AVS). PLoS One 11, e0154010.
Rezai-Zadeh K., Gate D., Szekely C. A. & Town T. (2009) Can peripheral leukocytes be used as Alzheimer's disease biomarkers? Expert Rev Neurother 9, 1623-33.
Richartz-Salzburger E., Batra A., Stransky E., Laske C., Kohler N., Bartels M., Buchkremer G. & Schott K. (2007) Altered lymphocyte distribution in Alzheimer's disease. J Psychiatr Res 41, 174-8.
Samieri C., Perier M. C., Gaye B., Proust-Lima C., Helmer C., Dartigues J. F., Berr C., Tzourio C. & Empana J. P. (2018) Association of Cardiovascular Health Level in Older Age With Cognitive Decline and Incident Dementia. JAMA 320, 657-64.
Schjeide B. M., McQueen M. B., Mullin K., DiVito J., Hogan M. F., Parkinson M., Hooli B., Lange C., Blacker D., Tanzi R. E. & Bertram L. (2009) Assessment of Alzheimer's disease case-control associations using family-based methods. Neurogenetics 10, 19-25.
Scuteri A., Coluccia R., Castello L., Nevola E., Brancati A. M. & Volpe M. (2009) Left ventricular mass increase is associated with cognitive decline and dementia in the elderly independently of blood pressure. Eur Heart J 30, 1525-9.
Sherva R., Baldwin C. T., Inzelberg R., Vardarajan B., Cupples L. A., Lunetta K., Bowirrat A., Naj A., Pericak-Vance M., Friedland R. P. & Farrer L. A. (2011) Identification of novel candidate genes for Alzheimer's disease by autozygosity mapping using genome wide SNP data. J Alzheimers Dis 23, 349-59.
Silverberg G. D., Mayo M., Saul T., Rubenstein E. & McGuire D. (2003) Alzheimer's disease, normal-pressure hydrocephalus, and senescent changes in CSF circulatory physiology: a hypothesis. Lancet Neurol 2, 506-11.
Song Y., Miyaki K., Suzuki T., Sasaki Y., Tsutsumi A., Kawakami N., Shimazu A., Takahashi M., Inoue A., Kan C., Kurioka S. & Shimbo T. (2014) Altered DNA methylation status of human brain derived neurotrophis factor gene could be useful as biomarker of depression. Am J Med Genet B Neuropsychiatr Genet 165B, 357-64.
Spina S. & Laws S. M. (2019) Insights into the pathogenesis of normal-pressure hydrocephalus. Neurology.
Takarada-Iemata M., Kezuka D., Takeichi T., Ikawa M., Hattori T., Kitao Y. & Hori O. (2014) Deletion of N-myc downstream-regulated gene 2 attenuates reactive astrogliosis and inflammatory response in a mouse model of cortical stab injury. J Neurochem 130, 374-87.
Tones V. I., Godoy J. A. & Inestrosa N. C. (2019) Modulating Wnt signaling at the root: Porcupine and Wnt acylation. Pharmacol Ther.
Town T., Tan J., Flavell R. A. & Mullan M. (2005) T-cells in Alzheimer's disease. Neuromolecular Med 7, 255-64.
Vallee A. & Lecarpentier Y. (2016) Alzheimer Disease: Crosstalk between the Canonical Wnt/Beta-Catenin Pathway and PPARs Alpha and Gamma. Front Neurosci 10, 459.
Wilhelm-Benartzi C. S., Koestler D. C., Karagas M. R., Flanagan J. M., Christensen B. C., Kelsey K. T., Marsit C. J., Houseman E. A. & Brown R. (2013) Review of processing and analysis methods for DNA methylation array data. Br J Cancer 109, 1394-402.
Winblad B., Amouyel P., Andrieu S., Ballard C., Brayne C., Brodaty H., Cedazo-Minguez A., Dubois B., Edvardsson D., Feldman H., Fratiglioni L., Frisoni G. B., Gauthier S., Georges J., Graff C., Iqbal K., Jessen F., Johansson G., Jonsson L., Kivipelto M., Knapp M., Mangialasche F., Melis R., Nordberg A., Rikkert M. O., Qiu C., Sakmar T. P., Scheltens P., Schneider L. S., Sperling R., Tjernberg L. O., Waldemar G., Wimo A. & Zetterberg H. (2016) Defeating Alzheimer's disease and other dementias: a priority for European science and society. Lancet Neurol 15, 455-532.
Wolfram J. A., Lesnefsky E. J., Hoit B. D., Smith M. A. & Lee H. G. (2011) Therapeutic potential of c-Myc inhibition in the treatment of hypertrophic cardiomyopathy. Ther Adv Chronic Dis 2, 133-44.
Wortmann M. (2012) Dementia: a global health priority—highlights from an ADI and World Health Organization report. Alzheimers Res Ther 4, 40.

What is claimed is:

1. A method for diagnosing Alzheimer's Disease or determining susceptibility to Alzheimer's Disease, the method comprising:
obtaining a blood sample from a target subject;
identifying the degree of methylation in one or a plurality of Alzheimer indicators genes in the blood sample, each Alzheimer indicators gene identified as being an indicator of the presence of or risk of developing Alzheimer's Disease, the plurality of Alzheimer indicators genes having been identified by a machine learning technique or by logistic regression; and
identifying the target subject as being at risk for Alzheimer's Disease if the amount of methylation of one or more Alzheimer indicator genes differs from the amount of methylation established in control subjects not having Alzheimer's Disease by a predetermined amount.

2. The method of claim 1 wherein the predetermined amount is at least a 30 percent difference in the amount of methylation as compared to control subjects.

3. The method of claim 1 further comprising treating the target subject for Alzheimer's Disease if the target subject is identified as being at risk.

4. The method of claim 1 further comprising treating the target subject for Alzheimer's Disease if the target subject is identified as being at risk in a clinical trial.

5. The method of claim 1 wherein the machine learning technique is a neural network method.

6. The method of claim 1 wherein the machine learning technique applies technique selected from the group consisting of a support vector machine (SVM), a Generalized linear Model (GLM), Prediction Analysis for Microarrays (PAM), Random Forest (RF) and Linear Discriminant Analysis (LDA).

7. The method of claim 1 wherein at least one gene in the plurality of Alzheimer indicators genes are hypomethylated in target subjects having or at risk for Alzheimer's Disease as compared to control subjects.

8. The method of claim 7 wherein the plurality of Alzheimer indicator genes include a least one or any combinations of PLVAP, KCNH2, TSTD3, SARM1, CTHRC1, TRAM1L1, GUSBL2, LOC731275, ZNF254 and TRIM6.

9. The method of claim 1 wherein at least one gene in the plurality of Alzheimer indicators genes are hypermethylated in target subjects having or at risk for Alzheimer's Disease as compared to control subjects.

10. The method of claim 9 wherein the plurality of Alzheimer indicator genes include a least one or any combinations of RNF5P1, RNF5, AGPAT1, GRB10, MIB2, WNT9B, MAF, THAP4, KCNK5 and KIF26A.

11. The method of claim 1 wherein the plurality of Alzheimer indicators genes includes a plurality genes listed in FIGS. 4 to 20.

12. The method of claim 1 wherein the plurality of Alzheimer indicators genes includes 1 to 80 percent of the genes listed in FIGS. 4 to 20.

13. The method of claim 1 wherein the plurality of Alzheimer indicators genes includes 10 to 50 percent of the genes listed in FIGS. 4 to 20.

14. The method of claim 1 wherein a risk of Alzheimer's Disease is calculated among patients having mild cognitive impairment.

15. A method for diagnosing Alzheimer's Disease or determining susceptibility to Alzheimer's Disease, the method comprising:
obtaining a blood sample from a target subject which can include blood spot on a filter paper obtained from a finger stick or blood drops from finger stick placed directly into a receptacle for subsequent DNA extraction;
performing gene methylation analysis of leucocytes in the blood sample; and
applying a trained neural network to determine if the target subject is at risk for or has Alzheimer's disease, the trained neural network having been trained from genome-wide methylation test sets that include a first group of testing subjects having Alzheimer's disease and a second group of test subjects not having Alzheimer's disease.

16. The method of claim 15 further comprising treating the target subject for Alzheimer's Disease if the target subject is identified as being at risk.

17. The method of claim 15 further comprising treating the target subject for Alzheimer's Disease if the target subject is identified as being at risk in a clinical trial.

18. The method of claim 15 wherein the gene methylation analysis is genome-wide. This includes any method of DNA methylation assessment genome-wide, up to and including methylation sequencing approaches.

19. The method of claim 15 wherein the gene methylation analysis is restricted to a plurality of previously identified Alzheimer indicator genes.

20. The method of claim 19 wherein at least one gene in the plurality of previously identified Alzheimer indicator genes are hypomethylated in target subjects having or at risk for Alzheimer's Disease as compared to control subjects.

21. The method of claim 19 wherein the target subject is identified as having or being at risk for or has Alzheimer's Disease if the amount of methylation for one or more genes in the plurality of previously identified Alzheimer indicator genes differs from the amount of methylation measured in control subjects not having Alzheimer's Disease by a predetermined amount.

22. The method of claim 21 wherein the predetermined amount is at least a 30 percent difference in the amount of methylation as compared to control subjections.

23. The method of claim 22 wherein the plurality of previously identified Alzheimer indicator genes includes a least one or any combinations of PLVAP, KCNH2, TSTD3, SARM1, CTHRC1, TRAM1L1, GUSBL2, LOC731275, ZNF254 and TRIM6.

24. The method of claim 19 wherein at least one gene of the plurality of previously identified Alzheimer indicator genes is hypermethylated in target subjects having or at risk for Alzheimer's Disease as compared to control subjects.

25. The method of claim 24 wherein the plurality of previously identified Alzheimer indicator genes includes a least one or any combinations of RNF5P1, RNF5, AGPAT1, GRB10, MIB2, WNT9B, MAF, THAP4, KCNK5 and KIF26A.

26. The method of claim 19 wherein the plurality of previously identified Alzheimer indicator genes includes a plurality genes listed in FIGS. 4 to 20.

27. The method of claim 19 wherein the plurality of previously identified Alzheimer indicator genes includes 1 to 80 percent of the genes listed in FIGS. 4 to 20.

28. The method of claim 19 wherein the plurality of previously identified Alzheimer indicator genes includes 10 to 50 percent of the genes listed in FIGS. 4 to 20.

29. An AI system for calculating risk of AD based on leucocyte DNA methylation analysis, the AI system comprising a computer processor executing the steps of
the method comprising:
obtaining a blood sample from a target subject;
identifying the degree of methylation in one or a plurality of Alzheimer indicators genes in the blood sample, each Alzheimer indicators gene identified as being an indicator of the presence of or risk of developing Alzheimer's Disease, the plurality of Alzheimer indicators genes having been identified by a machine learning technique or by logistic regression; and
identifying the target subject as being at risk for Alzheimer's Disease if the amount of methylation of one or more Alzheimer indicator genes differs from the amount of methylation established in control subjects not having Alzheimer's Disease by a predetermined amount.

30. The AI system of claim 29 wherein a risk of Alzheimer's Disease is calculated among patients having mild cognitive impairment.
